(19) United States
(12) Patent Application Publication
(10) Pub. No.: US 2024/0208889 A1
(43) Pub. Date: Jun. 27, 2024

(54) SOLID STATE FORMS OF TAPINAROF

(71) Applicant: TEVA PHARMACEUTICALS INTERNATIONAL GMBH

(72) Inventors: Polina Lapido, Rishon Lezion (IL); Doron Rudik, Modiin (IL)

(73) Assignee: TEVA PHARMACEUTICALS INTERNATIONAL GMBH, Jona (CH)

(21) Appl. No.: 17/925,858

(22) PCT Filed: May 19, 2021
(86) PCT No.: PCT/US21/33060

§ 371 Date: Nov. 17, 2022

Related U.S. Application Data
(60) Provisional application No. 63/026,821, filed on May 19, 2020.
(60) Provisional application No. 63/033,329, filed on Jun. 2, 2020.
(60) Provisional application No. 63/106,410, filed on Oct. 28, 2020.

(51) Int. Cl. C07C 39/21; A61K 31/05

(52) U.S. Cl. CPC C07C 39/21; A61K 31/05; C07B 2200/13

(57) ABSTRACT

The present disclosure encompasses solid state forms of Tapinarof, in embodiments crystalline polymorphs or co-crystals of Tapinarof, processes for preparation thereof, and pharmaceutical compositions thereof.

### FIELD OF THE DISCLOSURE

The present disclosure encompasses solid state forms of Tapinarof, in embodiments crystalline polymorphs or co-crystals of Tapinarof, processes for preparation thereof, and pharmaceutical compositions thereof.

### BACKGROUND OF THE DISCLOSURE

Tapinarof, 3,5-dihydroxy-4-isopropyl-trans-stilbene, has the following chemical structure:

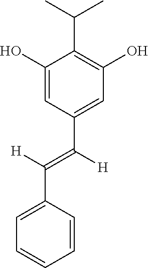 [SMILES: CC(C)c1c(O)cc(/C=C/c2ccccc2)cc1O]

Tapinarof is a therapeutic aryl hydrocarbon receptor modulating agent (TAMA), and it is developed for the treatment of psoriasis and atopic dermatitis.

The compound is described for example in Journal of Chemical Ecology, 7, 589-597 (1981), and International Publication Nos. WO 2001/042231 and WO 2004/031117. Crystalline forms of Tapinarof are disclosed in International Publication Nos. WO 2019/063002 and WO 2019/094934. International Publication No. WO 2019/094934 relates also to processes for preparation of Tapinarof.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Tapinarof.

### SUMMARY OF THE DISCLOSURE

The present disclosure provides crystalline polymorphs of Tapinarof and co-crystals of Tapinarof processes for preparation thereof, and pharmaceutical compositions thereof. These crystalline polymorphs and co-crystals can be used to prepare other solid state forms of Tapinarof.

The present disclosure also provides uses of any one of the said solid state forms of Tapinarof or co-crystals thereof, in the preparation of other solid state forms of Tapinarof.

The present disclosure provides crystalline polymorphs of Tapinarof or co-crystals thereof for use in medicine, including for the treatment of psoriasis and atopic dermatitis.

The present disclosure also encompasses the use of the crystalline polymorphs of Tapinarof and/or co-crystals thereof of the present disclosure for the preparation of pharmaceutical compositions and/or formulations.

In another aspect, the present disclosure provides pharmaceutical compositions comprising the crystalline polymorphs of Tapinarof or the co-crystals thereof according to the present disclosure.

The present disclosure includes processes for preparing the above mentioned pharmaceutical compositions. The processes include combining any one or more of the crystalline polymorphs of Tapinarof or the co-crystals thereof with at least one pharmaceutically acceptable excipient.

The crystalline polymorphs of Tapinarof or the co-crystals of Tapinarof as defined herein and the pharmaceutical compositions or formulations of the crystalline polymorphs of Tapinarof or the co-crystals thereof, may be used as medicaments, such as for the treatment of psoriasis and atopic dermatitis.

The present disclosure also provides methods of treating psoriasis and atopic dermatitis, by administering a therapeutically effective amount of any one or a combination of the crystalline polymorphs of Tapinarof or any one of the Tapinarof co-crystals of the present disclosure, or at least one of the above pharmaceutical compositions, to a subject suffering from psoriasis and atopic dermatitis, or otherwise in need of the treatment.

The present disclosure also provides uses of crystalline polymorphs of Tapinarof or of the co-crystals thereof of the present disclosure, or at least one of the above pharmaceutical compositions, for the manufacture of medicaments for treating e.g., psoriasis and atopic dermatitis.

### BRIEF DESCRIPTION OF THE DRAWINGS

FIG. 1 shows a characteristic X-ray powder diffraction pattern (XRPD) of Tapinarof Form T1, obtained by procedure A of example 1.

FIG. 2 shows X-ray powder diffraction pattern (XRPD) of Tapinarof nicotinamide Form NCC1, obtained by procedure A of example 2.

FIG. 3 shows X-ray powder diffraction pattern (XRPD) of Tapinarof nicotinamide Form NCC2, obtained by procedure A of example 3.

FIG. 4 shows X-ray powder diffraction pattern (XRPD) of Tapinarof proline Form PCC1, obtained by procedure A of example 4.

FIG. 5 shows X-ray powder diffraction pattern (XRPD) of Tapinarof piperazine Form PCC2, obtained by procedure A of example 5.

FIG. 6 shows X-ray powder diffraction pattern (XRPD) of Tapinarof form IV, obtained by reference example 1.

FIG. 7 shows X-ray powder diffraction pattern (XRPD) of Tapinarof Form T2.

FIG. 8 shows X-ray powder diffraction pattern (XRPD) of Tapinarof Form T3.

FIG. 9 shows X-ray powder diffraction pattern (XRPD) of Tapinarof Form T4.

FIG. 10 shows X-ray powder diffraction pattern (XRPD) of Tapinarof Form T5.

FIG. 11 shows X-ray powder diffraction pattern (XRPD) of Tapinarof Form T7.

FIG. 12 shows X-ray powder diffraction pattern (XRPD) of Tapinarof Form T9.

FIG. 13 shows X-ray powder diffraction pattern (XRPD) of Tapinarof form I, obtained by reference example 2.

FIG. 14 shows a characteristic solid state ¹³C NMR spectrum of Form NCC1 of Tapinarof nicotinamide co-crystal (full range 200-0 ppm).

FIG. 15 shows a characteristic solid state ¹³C NMR spectrum of Form NCC1 of Tapinarof nicotinamide co-crystal (100-0 ppm).

FIG. 16 shows a characteristic solid state ¹³C NMR spectrum of form NCC1 of Tapinarof nicotinamide co-crystal (200-100 ppm).

FIG. 17 shows a scanning electron microscopy (SEM) image of particles of NCC1 of Tapinarof nicotinamide co-crystal.

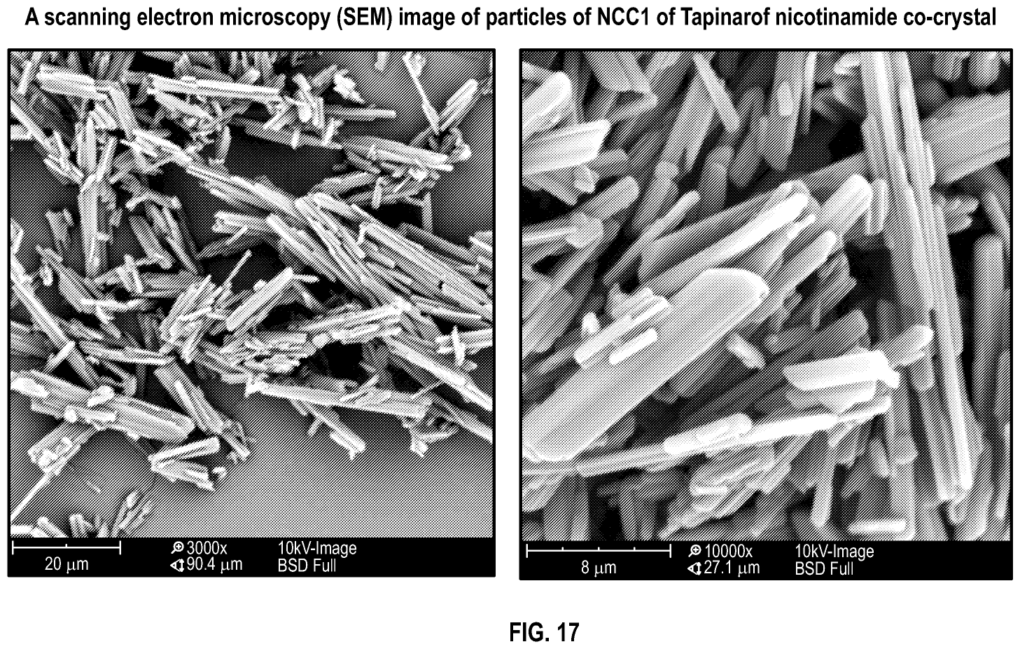

FIG. 18 shows the packing of NCC1 (Tapinarof nicotinamide (1:2)), view according a axis (Mercury).

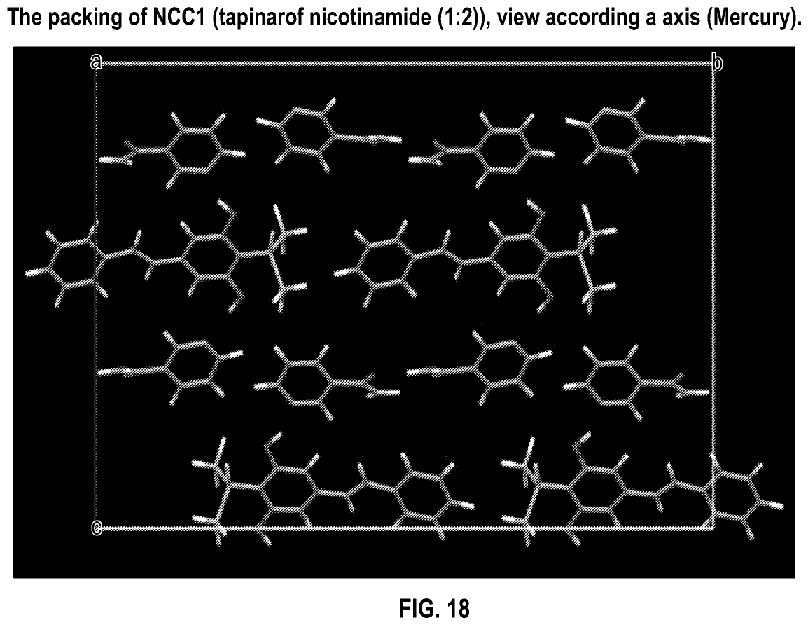

FIG. 19 shows a characteristic solid state ¹³C NMR spectrum of Form NCC2 of Tapinarof nicotinamide co-crystal (full range 200-0 ppm).

FIG. 20 shows a characteristic solid state ¹³C NMR spectrum of Form NCC2 of Tapinarof nicotinamide co-crystal (100-0 ppm).

FIG. 21 shows a characteristic solid state ¹³C NMR spectrum of form NCC2 of Tapinarof nicotinamide co-crystal (200-100 ppm).

FIG. 22 shows a scanning electron microscopy (SEM) image of particles of NCC2 of Tapinarof nicotinamide co-crystal.

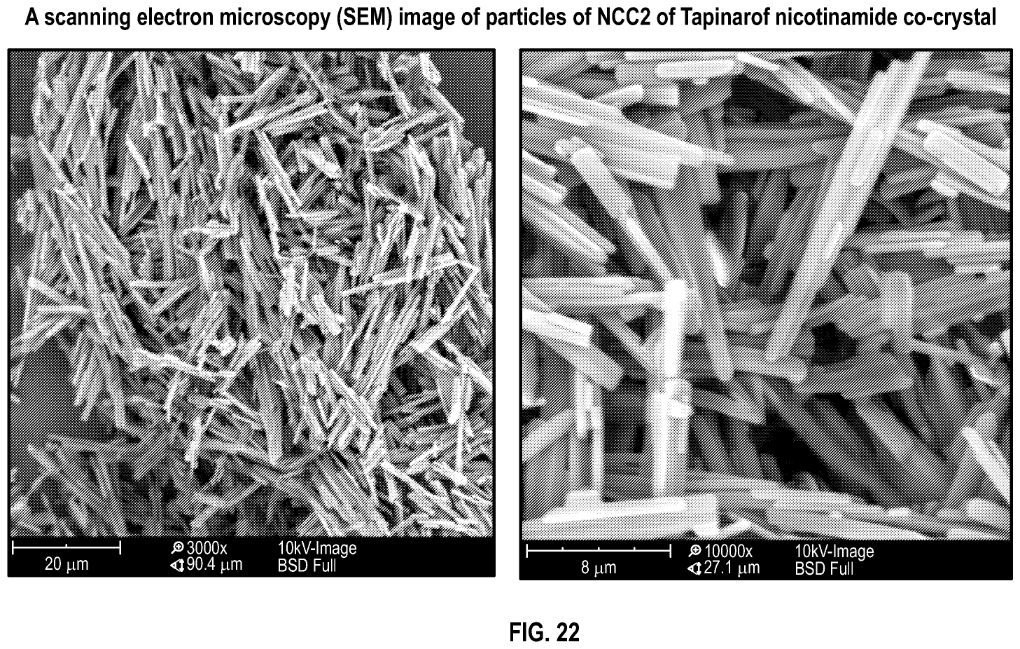

FIG. 23 shows the packing of NCC2 (Tapinarof nicotinamide (1:1)), view according a axis (Mercury).

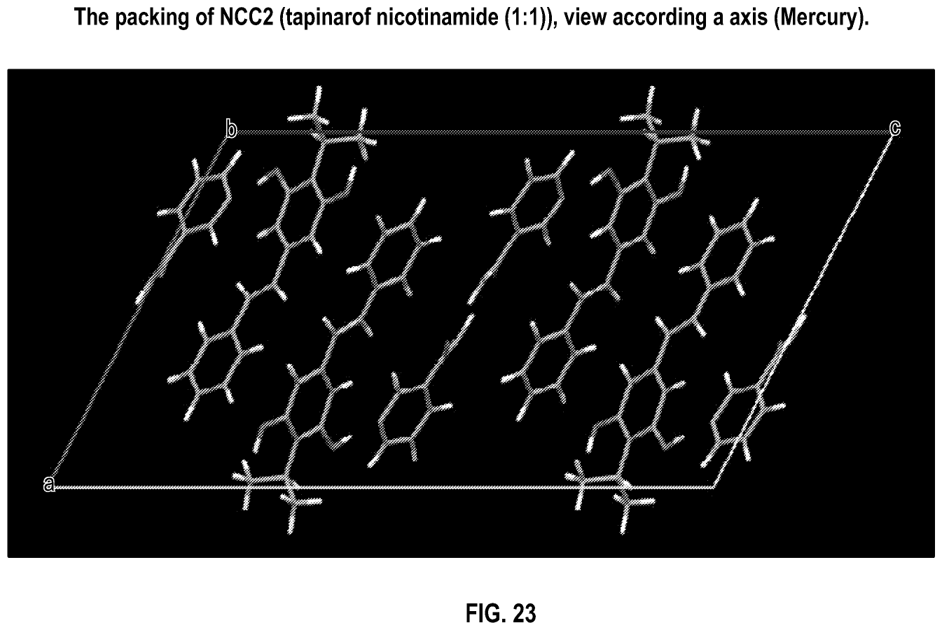

FIG. 24 shows a characteristic solid state ¹³C NMR spectrum of Form PCC1 of Tapinarof proline co-crystal (full range 200-0 ppm).

FIG. 25 shows a characteristic solid state ¹³C NMR spectrum of Form PCC1 of Tapinarof proline co-crystal (100-0 ppm).

FIG. 26 shows a characteristic solid state ¹³C NMR spectrum of form PCC1 of Tapinarof proline co-crystal (200-100 ppm).

FIG. 27 shows a scanning electron microscopy (SEM) image of particles of PCC1 of Tapinarof proline co-crystal.

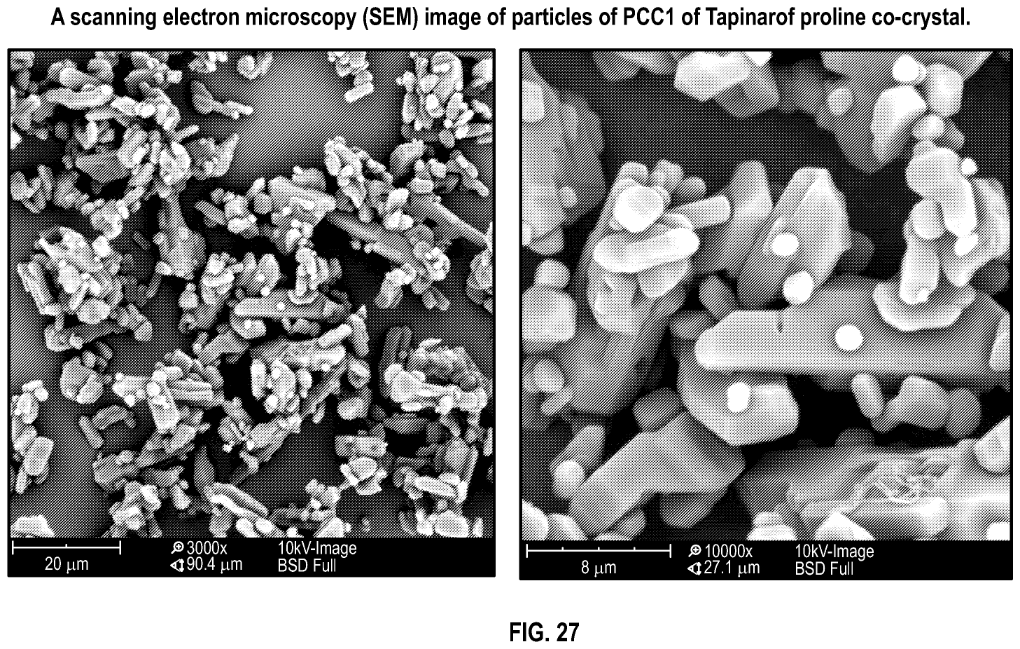

FIG. 28 shows the packing of PCC1 (Tapinarof L-proline (1:2)), view according a axis (Mercury).

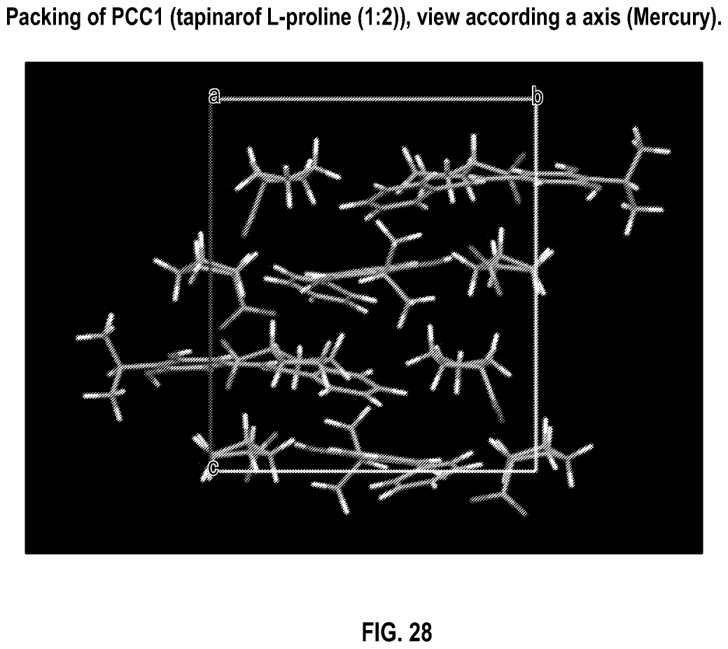

FIG. 29 shows a characteristic solid state ¹³C NMR spectrum of Form PCC2 of Tapinarof piperazine co-crystal (full range 200-0 ppm).

FIG. 30 shows a characteristic solid state ¹³C NMR spectrum of form PCC2 of Tapinarof piperazine co-crystal (200-100 ppm).

FIG. 31 shows a scanning electron microscopy (SEM) image of particles of PCC2 of Tapinarof piperazine co-crystal.

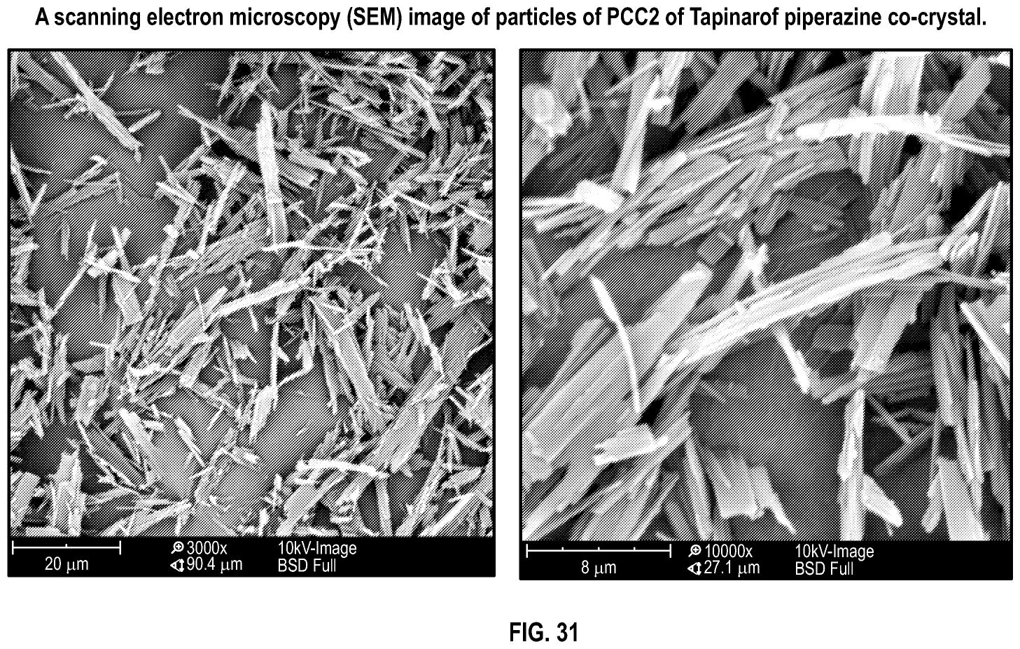

FIG. 32 shows the packing of PCC2 (Tapinarof piperazine (1:1)), view according a axis (Mercury).

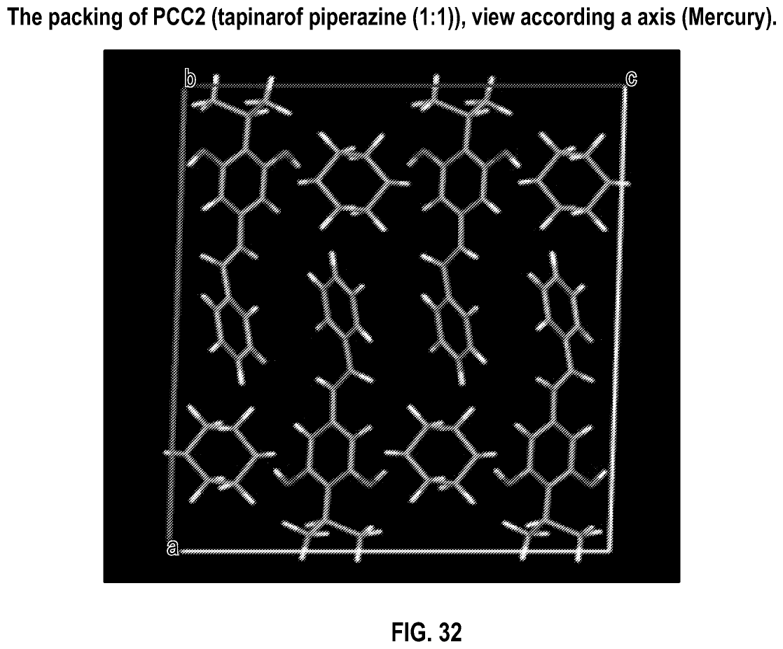

### DETAILED DESCRIPTION OF THE DISCLOSURE

The present disclosure encompasses solid state forms of Tapinarof and co-crystals of Tapinarof, processes for preparation thereof, and pharmaceutical compositions thereof.

Solid state properties of Tapinarof and crystalline polymorphs thereof can be influenced by controlling the conditions under which Tapinarof and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Tapinarof described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Tapinarof. In some embodiments of the disclosure, the described crystalline polymorph of Tapinarof may contain from about 1% to about 20% (w/w), from about 5% to about 20% (w/w), or from about 5% to about 10% (w/w) of one or more other crystalline polymorph of the same Tapinarof.

Depending on which other crystalline polymorphs a comparison is made, the crystalline polymorphs of Tapinarof of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Tapinarof referred to herein as being characterized by graphical data “as depicted in” or “as substantially depicted in” a Figure will thus be understood to include any crystal forms of Tapinarof characterized with the graphical data having such small variations, as are well known to the skilled person, in comparison with the Figure.

As used herein, and unless stated otherwise, the term “anhydrous” in relation to crystalline forms of Tapinarof, relates to a crystalline form of Tapinarof which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

“Co-Crystal” or “Co-crystal” as used herein is defined as a crystalline material including two or more molecules in the same crystalline lattice and associated by non-ionic and non-covalent bonds. In some embodiments, the co-crystal includes two molecules which are in natural state. As used herein, the term “isolated” in reference to crystalline polymorph of Tapinarof of the present disclosure corresponds to a crystalline polymorph of Tapinarof that is physically separated from the reaction mixture in which it is formed.

As used herein, unless stated otherwise, the XRPD measurements are taken using copper Kα radiation wavelength 1.5418 Å. XRPD peaks reported herein are measured using CuK α radiation, λ=1.5418 Å, typically at a temperature of 25±3° C.

As used herein, ¹³C NMR spectra are preferably measured at 11.7 T at magic angle spinning (MAS) frequency ωr/2π=64 kHz.

As used herein, unless stated otherwise, TGA analysis is carried out at a heating rate of 10° C./min to 250° C., preferably with a nitrogen flow of 40 ml/minute.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

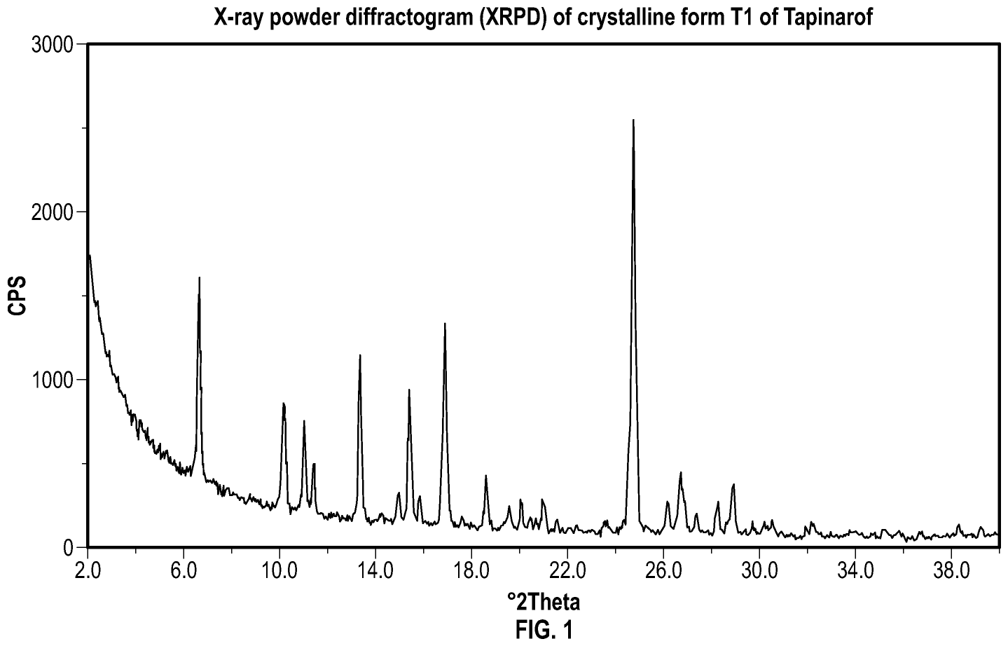

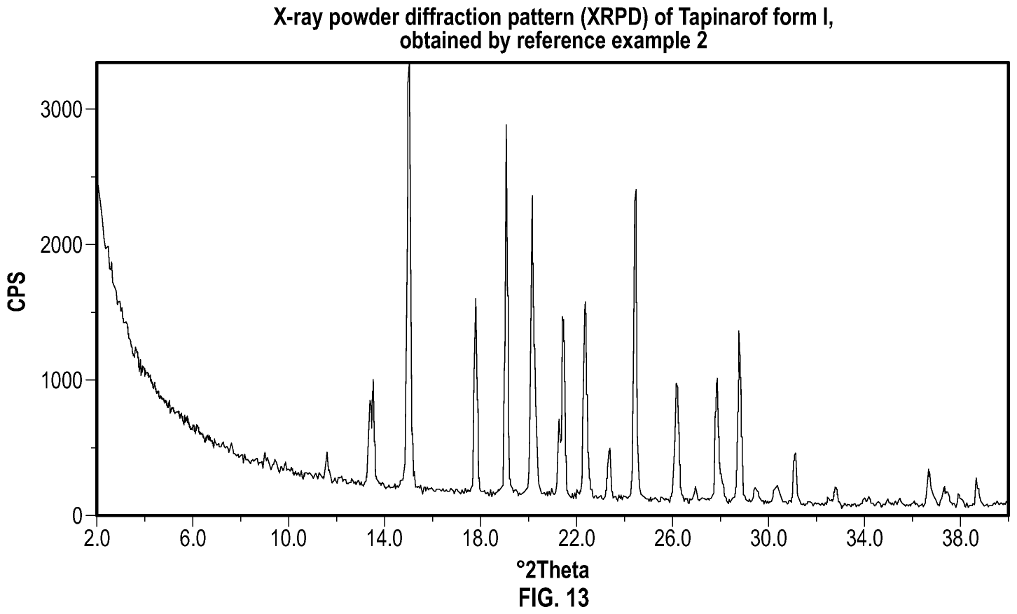

As used herein Form I of Tapinarof is as defined in International Publication No. WO 2019/063002. For example, according International Publication No. WO 2019/063002, Form I is defined as having characteristic XRPD peaks at 15.0, 19.0, 20.1, 21.4, 22.3 and 24.4 degrees 2-theta±0.2 degrees 2-theta. Form I is also defined therein as having characteristic XRPD peaks at 15.0, 17.8, 19.0, 20.1, 21.4, 22.3, 24.4, 26.8 and 27.8 degrees 2-theta±0.2 degrees 2-theta, or an X-ray powder diffraction pattern as shown in FIG. 1 therein. Form I of Tapinarof may alternatively be defined as having an XRPD substantially as depicted in FIG. 13 herein.

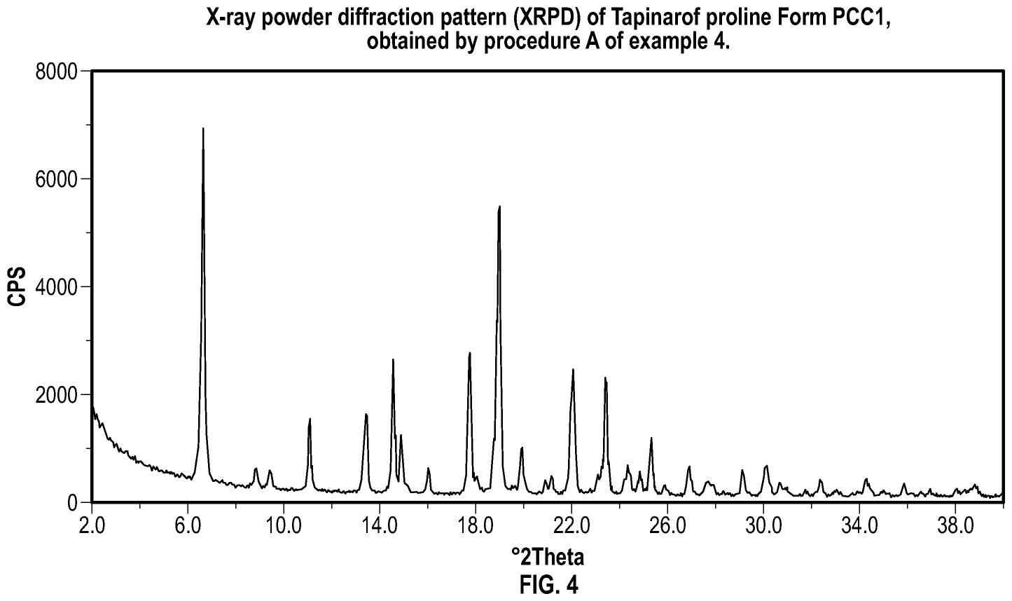

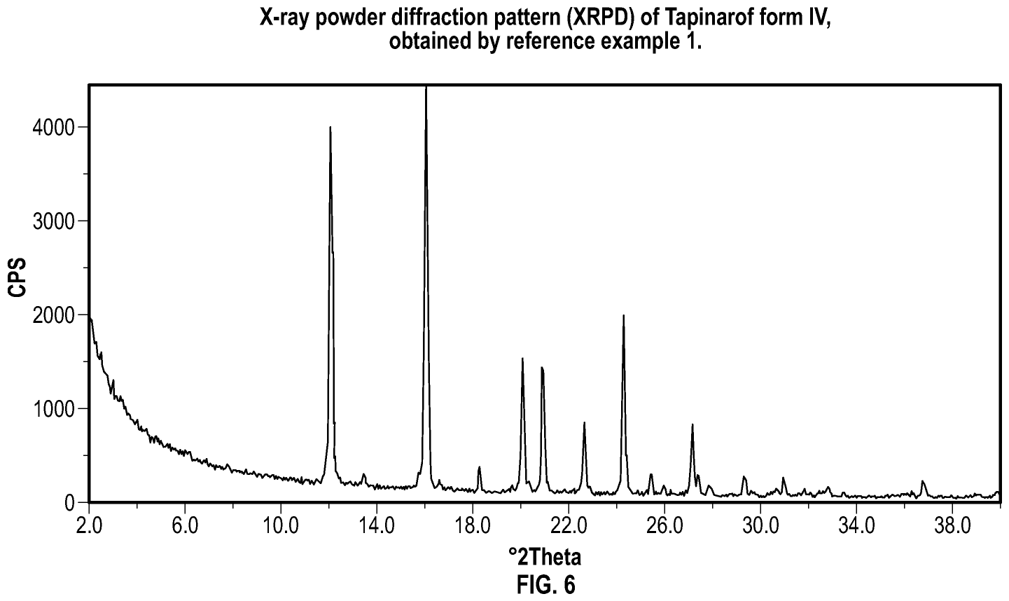

As used herein, Form IV of Tapinarof is as defined in International Publication No. WO 2019/063002, and is characterized by an XRPD having characteristic peaks at: 12.1, 13.3, 16.0, 20.0, 24.3, and 27.1 degrees 2-theta±0.2 degrees 2-theta. Form IV is also defined therein as having characteristic XRPD peaks at 12.1, 13.3, 16.0, 20.0, 20.9, 22.6, 24.3, 25.4, and 27.1 degrees 2-theta±0.2 degrees 2-theta, or an X-ray powder diffraction pattern as shown in FIG. 4 therein. Form IV of Tapinarof may alternatively be defined as having an XRPD substantially as depicted in FIG. 6 herein.

The present disclosure relates to Tapinarof in the form of propionic acid solvate.

The present disclosure includes a crystalline polymorph of Tapinarof, designated form T1. The crystalline Form T1 of Tapinarof may be characterized by data selected from one or more of the following: an X-ray powder diffraction pattern substantially as depicted in FIG. 1; an X-ray powder diffraction pattern having peaks at 6.7, 15.0, 15.4, 15.8 and 24.7 degrees 2-theta±0.2 degrees 2-theta; and combinations of these data.

Crystalline Form T1 of Tapinarof may be further characterized by an X-ray powder diffraction pattern having peaks at 6.7, 15.0, 15.4, 15.8 and 24.7 degrees 2-theta±0.2 degrees 2-theta, and also having any one, two, three, four or five additional peaks selected from 10.2, 11.4, 13.4, 16.9 and 18.6 degrees 2-theta±0.2 degrees 2-theta.

Crystalline Form T1 of Tapinarof may be alternatively characterized by an X-ray powder diffraction pattern having peaks at 6.7, 10.2, 11.4, 13.4, 15.0, 15.4, 15.8, 16.9, 18.6 and 24.7 degrees 2-theta±0.2 degrees 2-theta.

In one embodiment of the present disclosure, crystalline Form T1 of Tapinarof is isolated.

Crystalline Form T1 of Tapinarof may be a propionic acid solvate, preferably a mono propionic acid solvate. In embodiments, crystalline form T1 of Tapinarof may contain about 18% to about 26% of propionic acid, or about 22.5% of propionic acid by Weight.

Crystalline Form T1 of Tapinarof may be characterized by each of the above characteristics alone or by all possible combinations, e.g., an XRPD pattern having peaks at 6.7, 15.0, 15.4, 15.8 and 24.7 degrees 2-theta±0.2 degrees 2-theta; an XRPD pattern as depicted in FIG. 1, and combinations thereof.

In embodiments, crystalline form T1 may be polymorphically pure.

In embodiments, the propionic acid solvate of Tapinarof, preferably form T1 of Tapinarof, can be converted into other crystalline forms of Tapinarof or co-crystals of Tapinarof.

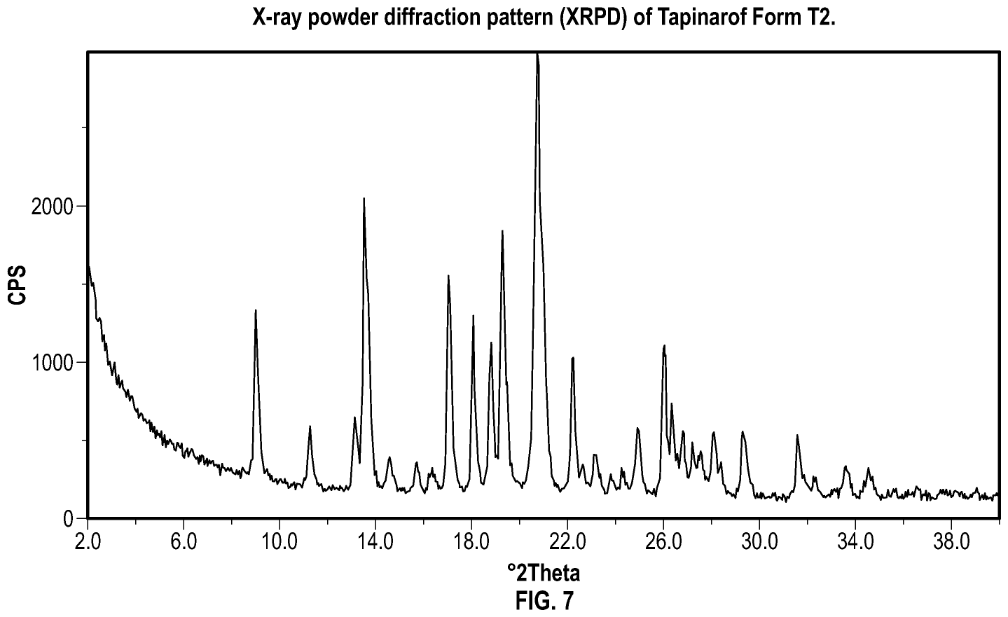

The present disclosure includes a crystalline polymorph of Tapinarof, designated form T2. The crystalline Form T2 of Tapinarof may be characterized by data selected from one or more of the following: an X-ray powder diffraction pattern substantially as depicted in FIG. 7; an X-ray powder diffraction pattern having peaks at 9.0, 11.3, 17.1, 19.3 and 20.8 degrees 2-theta±0.2 degrees 2-theta; and combinations of these data.

Crystalline Form T2 of Tapinarof may be further characterized by an X-ray powder diffraction pattern having peaks at 9.0, 11.3, 17.1, 19.3 and 20.8 degrees 2-theta±0.2 degrees 2-theta, and also having any one, two, three, four or five additional peaks selected from 13.6, 18.1, 18.8, 22.2 and 24.9 degrees 2-theta±0.2 degrees 2-theta.

Crystalline Form T2 of Tapinarof may be alternatively characterized by an X-ray powder diffraction pattern having peaks at 9.0, 11.3, 13.6, 17.1, 18.1, 18.8, 19.3, 20.8, 22.2, and 24.9 degrees 2-theta±0.2 degrees 2-theta.

In one embodiment of the present disclosure, crystalline Form T2 of Tapinarof is isolated.

Crystalline Form T2 of Tapinarof may be a DMSO solvate, preferably a mono DMSO solvate. In embodiments, crystalline form T2 of Tapinarof may contain about 19% to about 27% of DMSO, or about 23.5% of DMSO by Weight.

Crystalline Form T2 of Tapinarof may be characterized by each of the above characteristics alone or by all possible combinations, e.g., an XRPD pattern having peaks at 9.0, 11.3, 17.1, 19.3 and 20.8 degrees 2-theta±0.2 degrees 2-theta; an XRPD pattern as depicted in FIG. 7, and combinations thereof.

The present disclosure relates to Tapinarof in the form of N-methyl-pyrrolidone solvate.

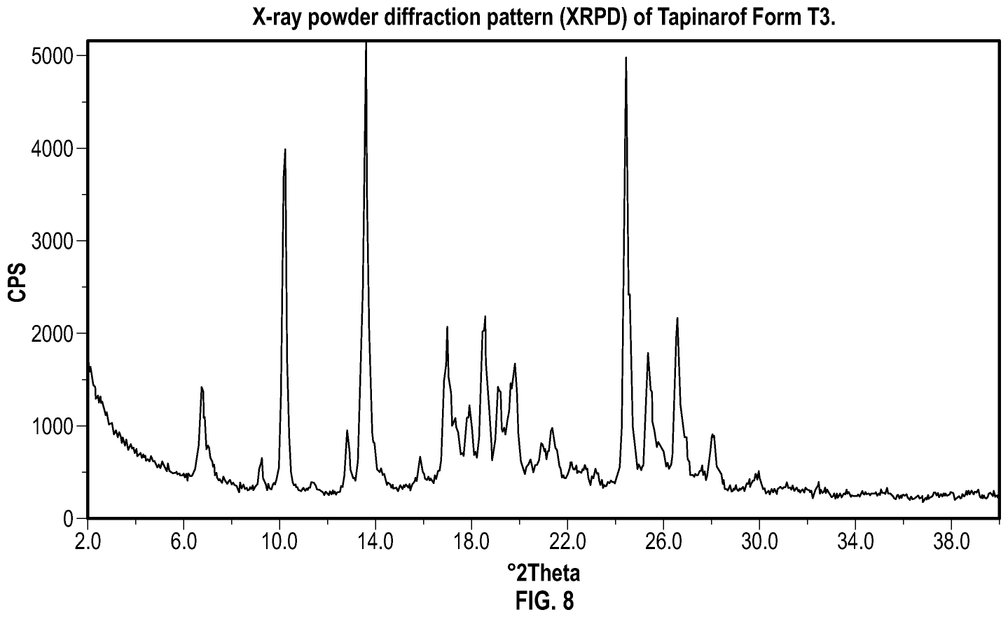

The present disclosure includes a crystalline polymorph of Tapinarof, designated form T3. The crystalline Form T3 of Tapinarof may be characterized by data selected from one or more of the following: an X-ray powder diffraction pattern substantially as depicted in FIG. 8; an X-ray powder diffraction pattern having peaks at 6.8, 10.3, 13.6, 18.5 and 24.4 degrees 2-theta±0.2 degrees 2-theta; and combinations of these data.

Crystalline Form T3 of Tapinarof may be further characterized by an X-ray powder diffraction pattern having peaks at 6.8, 10.3, 13.6, 18.5 and 24.4 degrees 2-theta±0.2 degrees 2-theta, and also having any one, two, three, four or five additional peaks selected from 9.3, 12.8, 17.0, 25.4 and 26.5 degrees 2-theta±0.2 degrees 2-theta.

Crystalline Form T3 of Tapinarof may be alternatively characterized by an X-ray powder diffraction pattern having peaks at 6.8, 9.3, 10.3, 12.8, 13.6, 17.0, 18.5, 24.4, 25.4, and 26.5 degrees 2-theta±0.2 degrees 2-theta.

In one embodiment of the present disclosure, crystalline Form T3 of Tapinarof is isolated.

Crystalline Form T3 of Tapinarof may be a NMP (N-Methyl-2-pyrrolidone) solvate, preferably a hemi N-methyl-2-pyrrolidone solvate. In embodiments, crystalline form T3 of Tapinarof may contain about 9% to about 15% of-methyl-2-pyrrolidone solvate, or about 12% of-methyl-2-pyrrolidone solvate by weight.

Crystalline Form T3 of Tapinarof may be characterized by each of the above characteristics alone or by all possible combinations, e.g., an XRPD pattern having peaks at 6.8, 10.3, 13.6, 18.5 and 24.4 degrees 2-theta±0.2 degrees 2-theta; an XRPD pattern as depicted in FIG. 8, and combinations thereof.

The present disclosure relates to Tapinarof in the form of a cyclohexanone solvate.

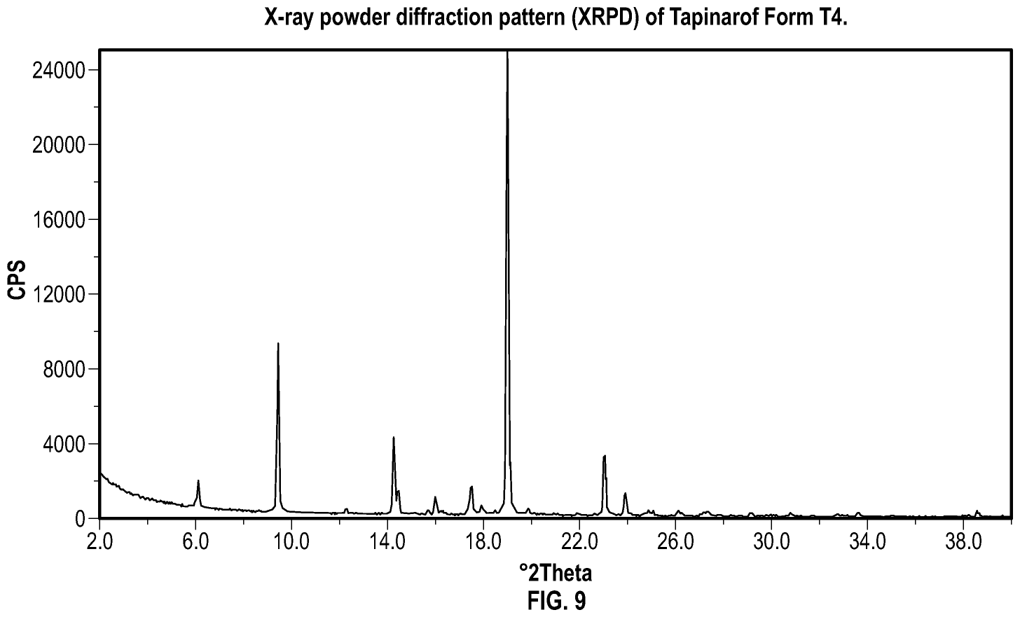

The present disclosure includes a crystalline polymorph of Tapinarof, designated form T4. The crystalline Form T4 of Tapinarof may be characterized by data selected from one or more of the following: an X-ray powder diffraction pattern substantially as depicted in FIG. 9; an X-ray powder diffraction pattern having peaks at 6.2, 9.5, 14.3, 19.0 and 23.1 degrees 2-theta±0.2 degrees 2-theta; and combinations of these data.

Crystalline Form T4 of Tapinarof may be further characterized by an X-ray powder diffraction pattern having peaks at 6.2, 9.5, 14.3, 19.0 and 23.1 degrees 2-theta±0.2 degrees 2-theta, and also having any one, two, three, four or five additional peaks selected from 12.3, 16.0, 17.5, 17.9 and 23.9 degrees 2-theta±0.2 degrees 2-theta.

Crystalline Form T4 of Tapinarof may be alternatively characterized by an X-ray powder diffraction pattern having peaks at 6.2, 9.5, 12.3, 14.3, 16.0, 17.5, 17.9, 19.0, 23.1 and 23.9 degrees 2-theta±0.2 degrees 2-theta.

In one embodiment of the present disclosure, crystalline Form T4 of Tapinarof is isolated.

Crystalline Form T4 of Tapinarof may be a cyclohexanone solvate, preferably a monocyclohexanone solvate. In embodiments, crystalline form T4 of Tapinarof may contain about 20% to about 30% of cyclohexanone, or about 24% of cyclohexanone by weight.

In embodiments crystalline form T4 of Tapinarof may be polymorphically pure.

Crystalline Form T4 of Tapinarof may be characterized by each of the above characteristics alone or by all possible combinations, e.g., an XRPD pattern having peaks at 6.2, 9.5, 14.3, 19.0 and 23.1 degrees 2-theta±0.2 degrees 2-theta; an XRPD pattern as depicted in FIG. 9, and combinations thereof.

The present disclosure relates to Tapinarof in the form of acetyl acetone solvate.

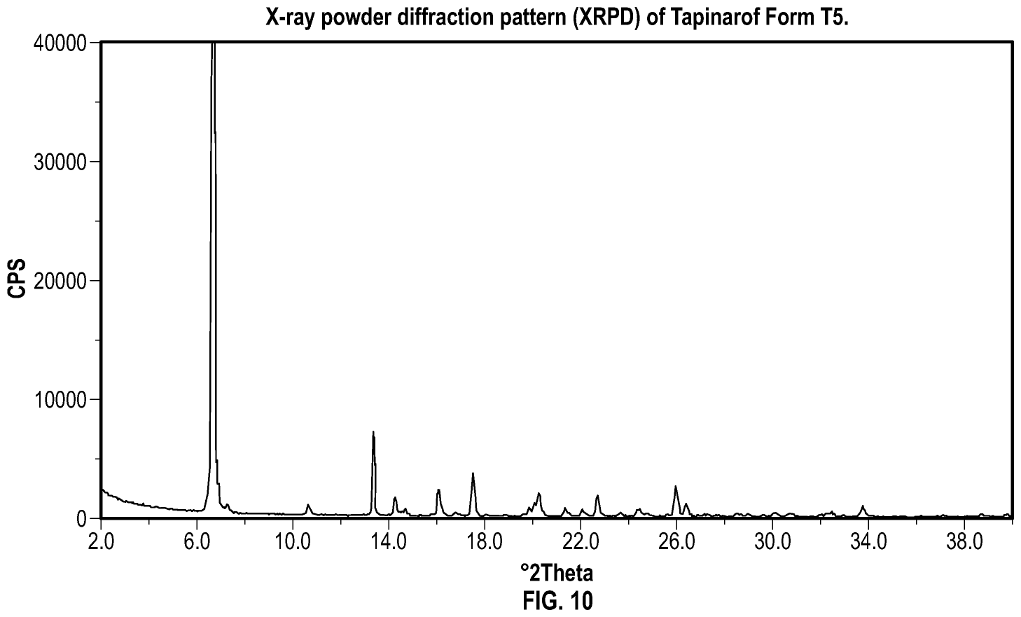

The present disclosure includes a crystalline polymorph of Tapinarof, designated form T5. The crystalline Form T5 of Tapinarof may be characterized by data selected from one or more of the following: an X-ray powder diffraction pattern substantially as depicted in FIG. 10; an X-ray powder diffraction pattern having peaks at 6.6, 13.3, 16.1, 17.5 and 22.7 degrees 2-theta±0.2 degrees 2-theta; and combinations of these data.

Crystalline Form T5 of Tapinarof may be further characterized by an X-ray powder diffraction pattern having peaks at 6.6, 13.3, 16.1, 17.5 and 22.7 degrees 2-theta±0.2 degrees 2-theta, and also having any one, two, three, four or five additional peaks selected from 10.6, 14.2, 20.2, 26.0 and 26.4 degrees 2-theta±0.2 degrees 2-theta.

Crystalline Form T5 of Tapinarof may be further characterized by an X-ray powder diffraction pattern having peaks at 6.6, 10.6, 13.3, 14.2, 16.1, 17.5, 20.2, 22.7, 26.0, and 26.4 degrees 2-theta±0.2 degrees 2-theta.

In one embodiment of the present disclosure, crystalline Form T5 of Tapinarof is isolated.

Crystalline Form T5 of Tapinarof may be an Acetyl acetone solvate.

Crystalline Form T5 of Tapinarof may be characterized by each of the above characteristics alone or by all possible combinations, e.g., an XRPD pattern having peaks at 6.6, 13.3, 16.1, 17.5 and 22.7 degrees 2-theta±0.2 degrees 2-theta; an XRPD pattern as depicted in FIG. 10, and combinations thereof.

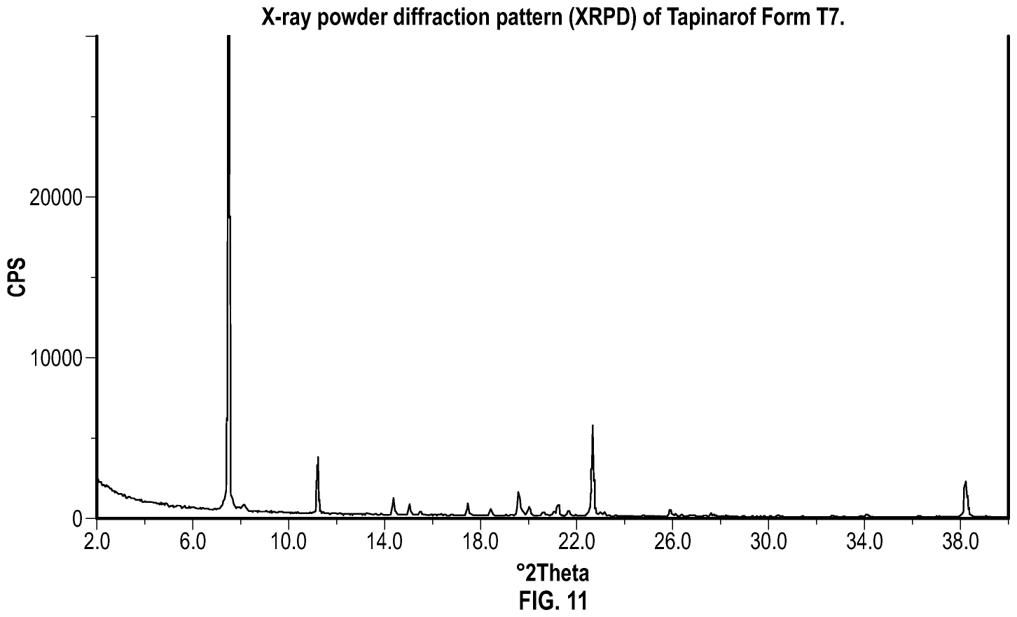

The present disclosure includes a crystalline polymorph of Tapinarof, designated form T7. The crystalline Form T7 of Tapinarof may be characterized by data selected from one or more of the following: an X-ray powder diffraction pattern substantially as depicted in FIG. 11; an X-ray powder diffraction pattern having peaks at 7.5, 11.2, 18.4, 19.6 and 22.7 degrees 2-theta±0.2 degrees 2-theta; and combinations of these data.

Crystalline Form T7 of Tapinarof may be further characterized by an X-ray powder diffraction pattern having peaks at 7.5, 11.2, 18.4, 19.6 and 22.7 degrees 2-theta±0.2 degrees 2-theta, and also having any one, two, three, four or five additional peaks selected from 14.4, 15.0, 17.4, 20.0 and 20.6 degrees 2-theta±0.2 degrees 2-theta.

Crystalline Form T7 of Tapinarof may be further characterized by an X-ray powder diffraction pattern having peaks at 7.5, 11.2, 14.4, 15.0, 17.4, 18.4, 19.6, 20.0, 20.6, and 22.7 degrees 2-theta±0.2 degrees 2-theta.

In one embodiment of the present disclosure, crystalline Form T7 of Tapinarof is isolated.

Crystalline Form T7 of Tapinarof may be a DMSO solvate.

Crystalline Form T7 of Tapinarof may be characterized by each of the above characteristics alone or by all possible combinations, e.g., an XRPD pattern having peaks at 7.5, 11.2, 18.4, 19.6 and 22.7 degrees 2-theta±0.2 degrees 2-theta; an XRPD pattern as depicted in FIG. 11, and combinations thereof.

The present disclosure relates to Tapinarof in the form of isoamyl alcohol solvate.

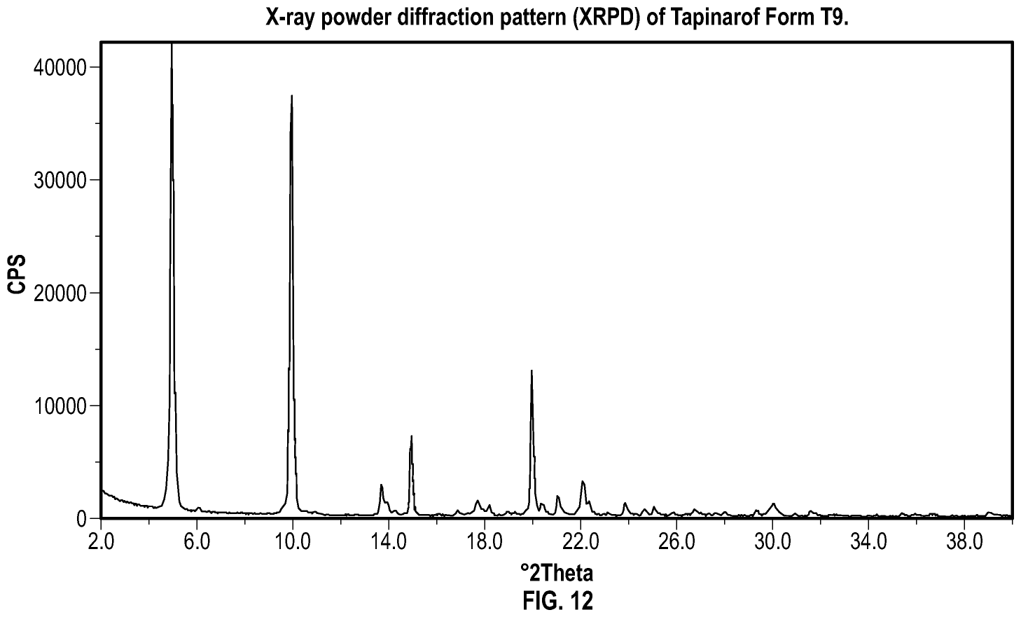

The present disclosure includes a crystalline polymorph of Tapinarof, designated form T9. The crystalline Form T9 of Tapinarof may be characterized by data selected from one or more of the following: an X-ray powder diffraction pattern substantially as depicted in FIG. 12; an X-ray powder diffraction pattern having peaks at 4.9, 10.0, 13.7, 20.0 and 21.0 degrees 2-theta±0.2 degrees 2-theta; and combinations of these data.

Crystalline Form T9 of Tapinarof may be further characterized by an X-ray powder diffraction pattern having peaks at 4.9, 10.0, 13.7, 20.0 and 21.0 degrees 2-theta±0.2 degrees 2-theta, and also having any one, two, three, four or five additional peaks selected from 14.9, 17.7, 20.4, 22.1 and 23.9 degrees 2-theta±0.2 degrees 2-theta.

Crystalline Form T9 of Tapinarof may be alternatively characterized by an X-ray powder diffraction pattern having peaks at 4.9, 10.0, 13.7, 14.9, 17.7, 20.0, 20.4, 21.0, 22.1, and 23.9 degrees 2-theta±0.2 degrees 2-theta.

In one embodiment of the present disclosure, crystalline Form T9 of Tapinarof is isolated.

Crystalline Form T9 of Tapinarof may be an Isoamyl alcohol solvate, preferably a hemi isoamyl alcohol solvate. In embodiments, crystalline form T9 of Tapinarof may contain about 10% to about 18% of isoamyl alcohol, or about 15% of isoamyl alcohol by Weight.

In embodiments crystalline form T9 of Tapinarof may be polymorphically pure.

Crystalline Form T9 of Tapinarof may be characterized by each of the above characteristics alone or by all possible combinations, e.g., an XRPD pattern having peaks at 4.9, 10.0, 13.7, 20.0 and 21.0 degrees 2-theta±0.2 degrees 2-theta; an XRPD pattern as depicted in FIG. 12, and combinations thereof.

The present disclosure relates to Tapinarof nicotinamide co crystal.

The present disclosure further encompasses Tapinarof nicotinamide co-crystal designated form NCC1. In embodiments the molar ratio of nicotinamide and Tapinarof in form NCC1 is typically about 2.

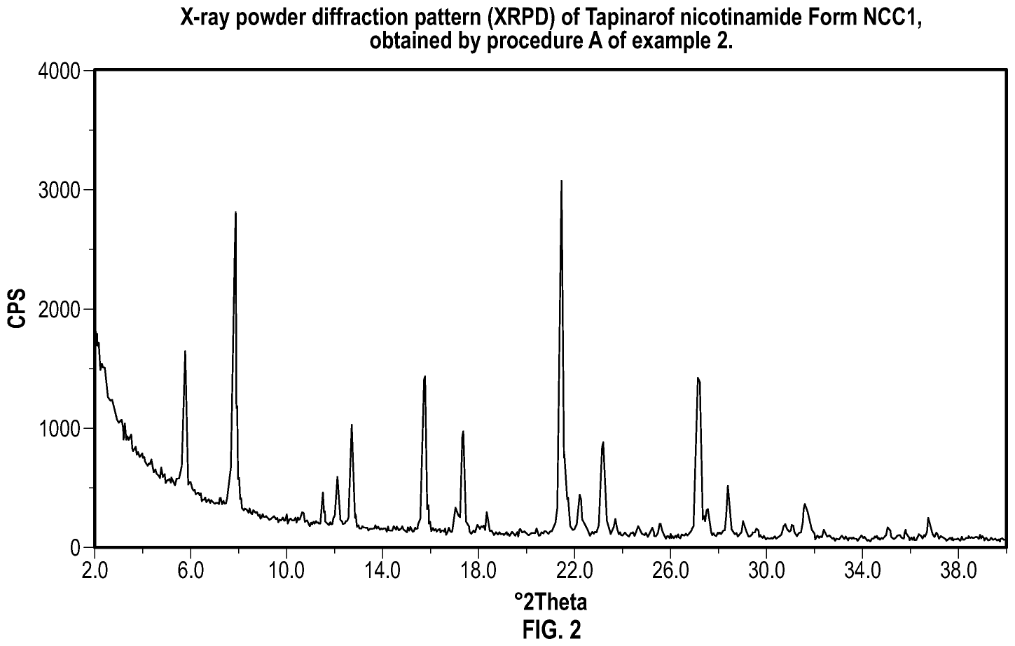

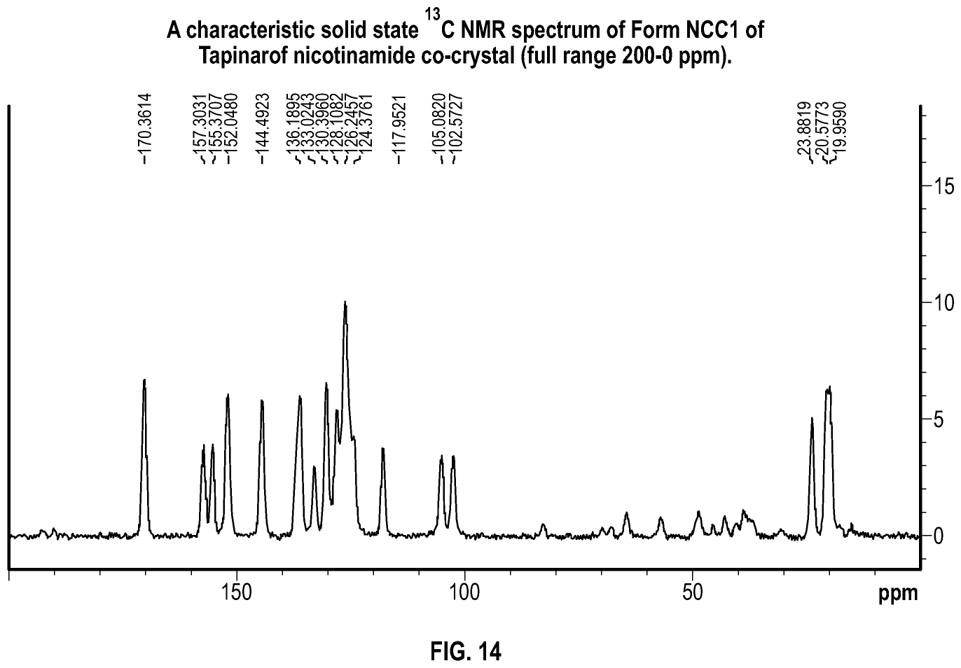

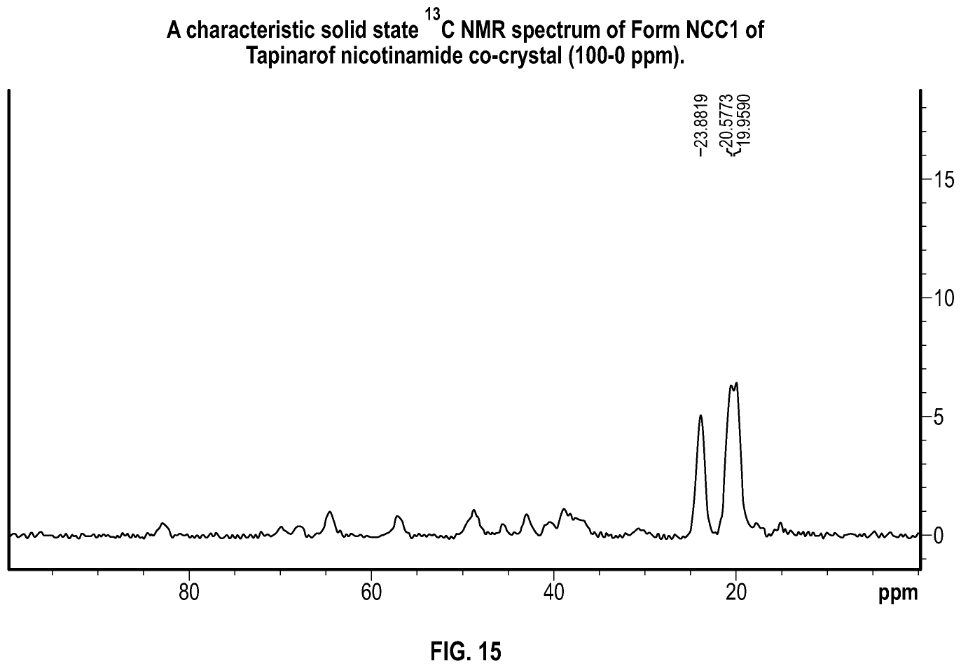

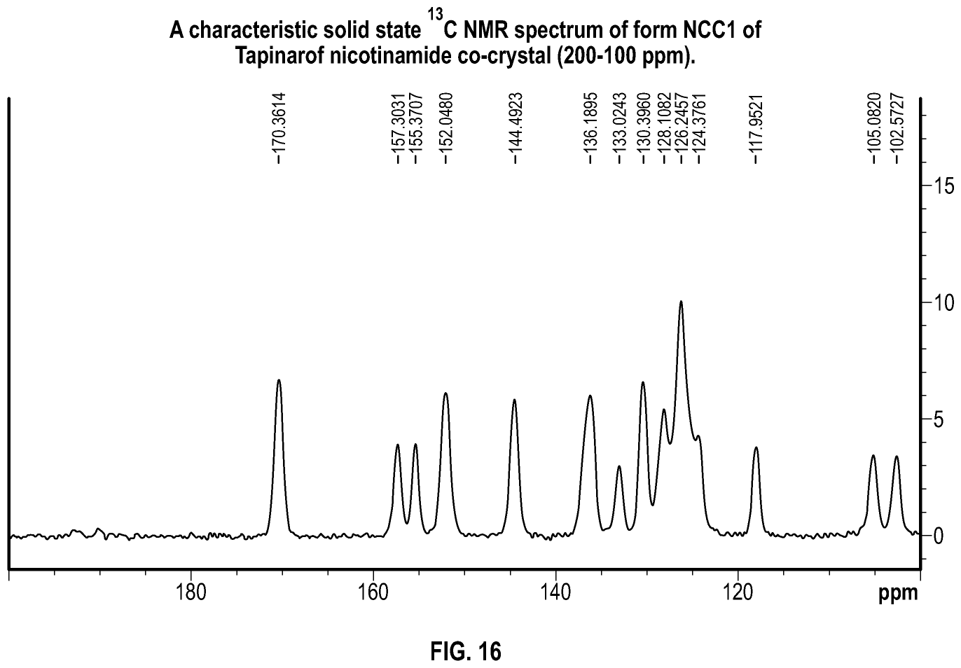

Crystalline form NCC1 of Tapinarof nicotinamide may be characterized by data selected from one or more of the following: an X-ray powder diffraction pattern substantially as depicted in FIG. 2; an X-ray powder diffraction pattern having peaks at 5.8, 7.9, 12.7, 15.8 and 21.4 degrees 2-theta±0.2 degrees 2-theta; a solid state ¹³C NMR spectrum with characteristic peaks at 152.1, 144.5, 136.2, 118.0 and 105.1 ppm±2 ppm; a solid state ¹³C NMR spectrum having the following chemical shift absolute differences from reference peak at 170.4 ppm±1 ppm: 18.3, 25.9, 34.2, 52.4 and 65.3 ppm±1 ppm; a solid state ¹³C NMR spectrum substantially as depicted in any of FIGS. 14, 15 and 16; and combinations of these data.

Crystalline Form NCC1 of Tapinarof nicotinamide may be further characterized by an X-ray powder diffraction pattern having peaks at 5.8, 7.9, 12.7, 15.8 and 21.4 degrees 2-theta 0.2 degrees 2-theta, and also having any one, two, three, four or five additional peaks selected from 11.5, 12.1, 17.4, 23.3 and 27.2 degrees 2-theta±0.2 degrees 2-theta.

Crystalline form NCC1 of Tapinarof nicotinamide may be alternatively characterized by an X-ray powder diffraction pattern having peaks at 5.8, 7.9, 11.5, 12.1, 12.7, 15.8, 17.4, 21.4, 23.3 and 27.2 degrees 2-theta±0.2 degrees 2-theta.

Alternatively, or in addition to the above, form NCC1 of Tapinarof nicotinamide co-crystal may be characterized by the following unit cell data:
a=5.1850 Å
b=30.6936 Å
c=16.5912 Å
α=90°
β=92.2897°
γ=90°
cell_volume 2638.3 Å³
symmetry cell setting: monoclinic
Space group Cc.

In one embodiment of the present disclosure, crystalline Form NCC1 of Tapinarof nicotinamide is isolated.

Crystalline NCC1 of Tapinarof nicotinamide co crystal may be characterized by each of the above characteristics alone or by all possible combinations, e.g., an XRPD pattern having peaks at 5.8, 7.9, 12.7, 15.8 and 21.4 degrees 2-theta±0.2 degrees 2-theta; an XRPD pattern as depicted in FIG. 2, and combinations of these data.

The present disclosure further encompasses Tapinarof nicotinamide co-crystal designated form NCC2. In embodiments the molar ratio of nicotinamide and Tapinarof in form NCC2 is typically about 1.

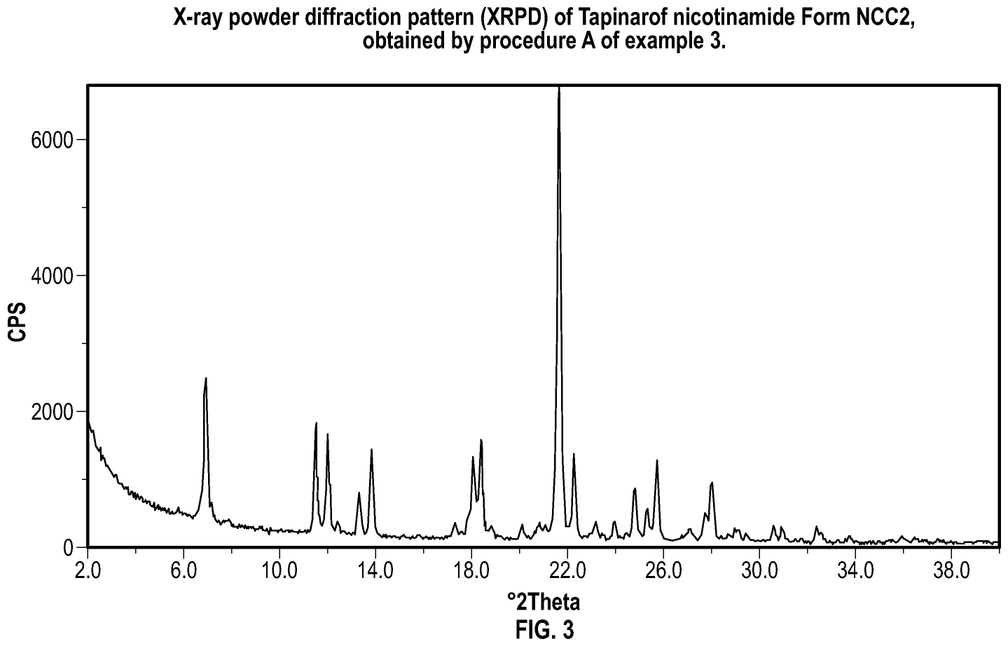

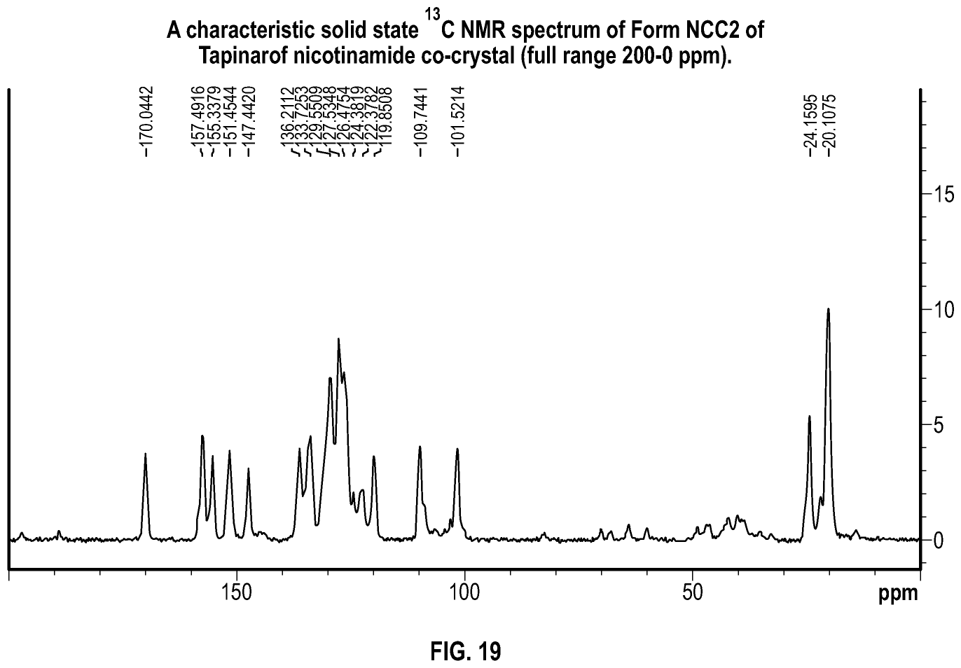

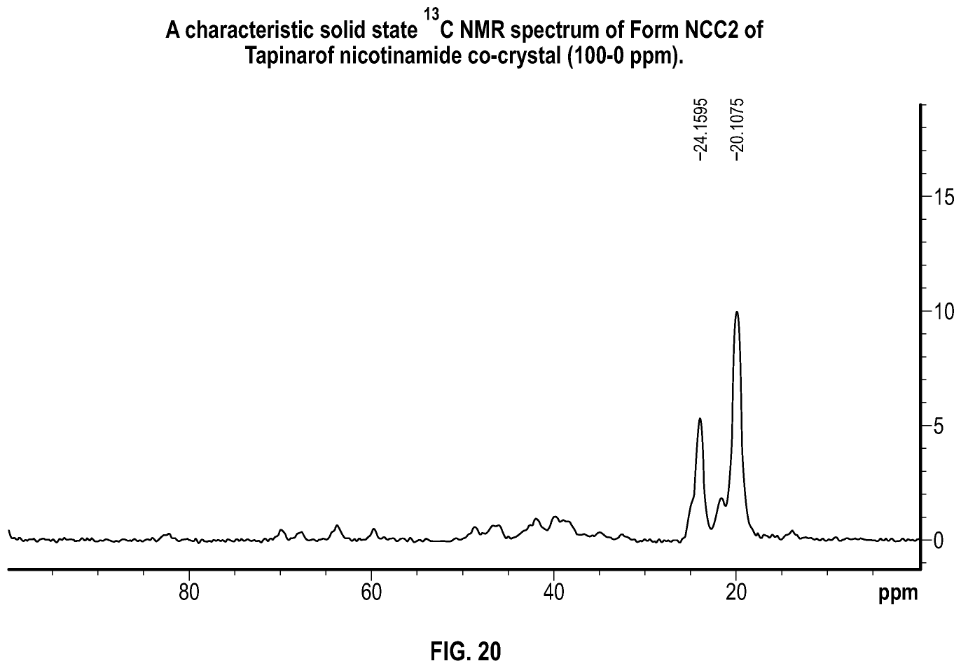

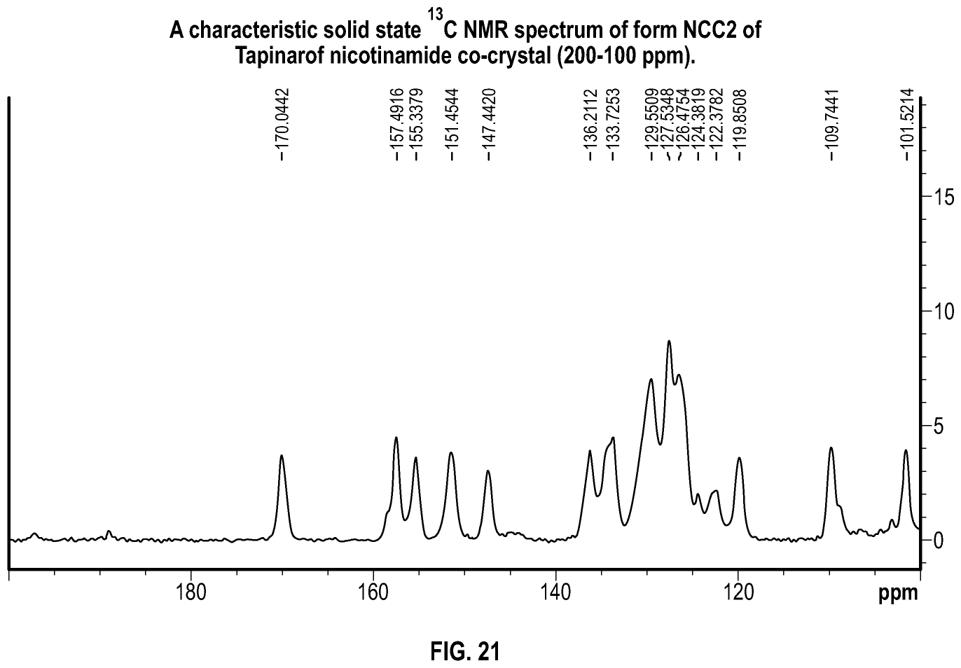

Crystalline form NCC2 of Tapinarof nicotinamide may be characterized by data selected from one or more of the following: an X-ray powder diffraction pattern substantially as depicted in FIG. 3; an X-ray powder diffraction pattern having peaks at 6.9, 13.3, 13.8, 21.6 and 22.2 degrees 2-theta±0.2 degrees 2-theta; a solid state ¹³C NMR spectrum with characteristic peaks at 151.5, 147.4, 119.8, 109.7 and 101.5 ppm±2 ppm; a solid state ¹³C NMR spectrum having the following chemical shift absolute differences from reference peak at 170.0 ppm±1 ppm: 18.5, 22.6, 50.2, 60.3 and 68.5 ppm±1 ppm; a solid state ¹³C NMR spectrum substantially as depicted in any of FIGS. 19, 20 and 21; and combinations of these data.

Crystalline Form NCC2 of Tapinarof nicotinamide may be further characterized by an X-ray powder diffraction pattern having peaks at 6.9, 13.3, 13.8, 21.6 and 22.2 degrees 2-theta 0.2 degrees 2-theta, and also having any one, two, three, four or five additional peaks selected from 12.0, 18.0, 18.3, 24.8 and 25.7 degrees 2-theta±0.2 degrees 2-theta.

Crystalline form NCC2 of Tapinarof nicotinamide may be alternatively characterized by an X-ray powder diffraction pattern having peaks at 6.9, 12.0, 13.3, 13.8, 18.0, 18.3, 21.6, 22.2, 24.8 and 25.7 degrees 2-theta±0.2 degrees 2-theta.

Alternatively, or in addition to the above, form NCC2 of Tapinarof nicotinamide co-crystal may be characterized by the following unit cell data:
a=15.4014 Å
b=5.2492 Å
c=28.5194 Å
α=90°
β=120.1912°
γ=900
cell_volume 1992.9 Å³
symmetry cell setting: monoclinic
Space group P 2₁/c.

In one embodiment of the present disclosure, crystalline Form NCC2 of Tapinarof nicotinamide is isolated.

Crystalline NCC2 of Tapinarof nicotinamide co crystal may be characterized by each of the above characteristics alone or by all possible combinations, e.g., an XRPD pattern having peaks at 6.9, 13.3, 13.8, 21.6 and 22.2 degrees 2-theta±0.2 degrees 2-theta; an XRPD pattern as depicted in FIG. 3, and combinations of these data.

The present disclosure further encompasses Tapinarof proline co-crystal designated form PCC1. In embodiments the molar ratio of proline to Tapinarof in form PCC1 is typically about 2.

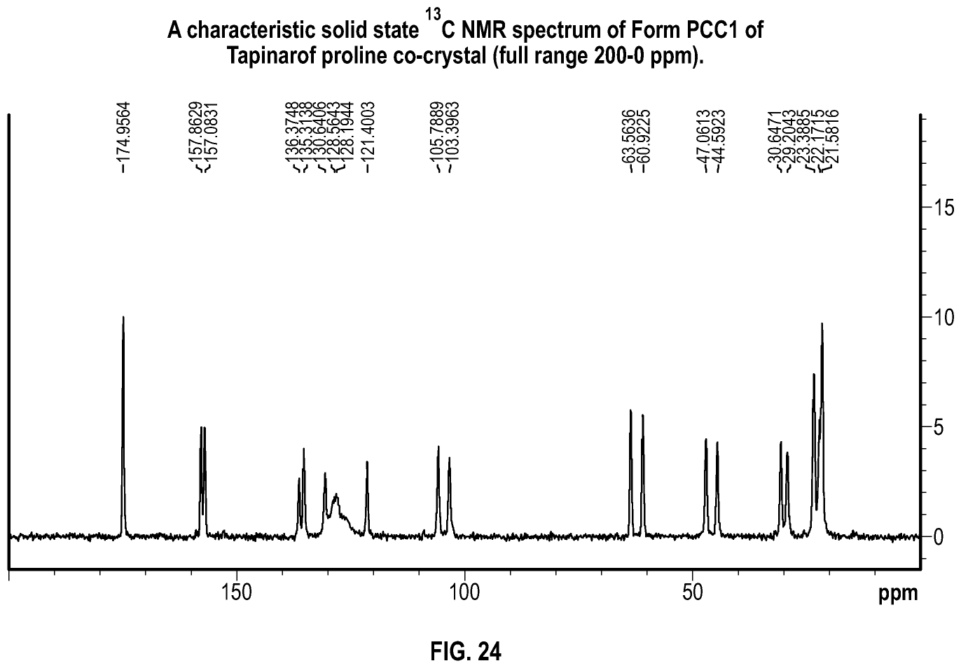

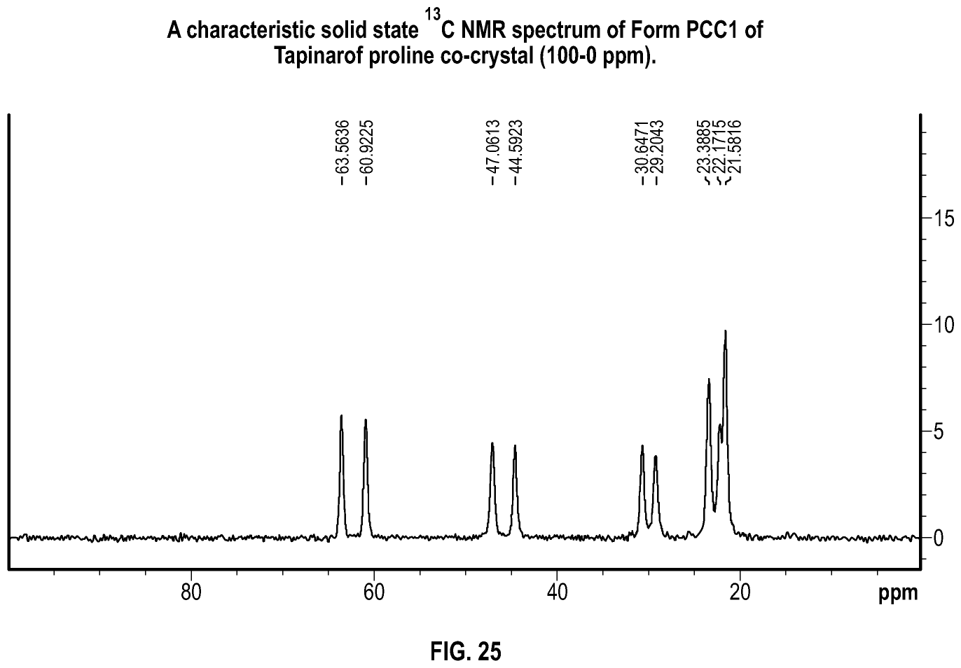

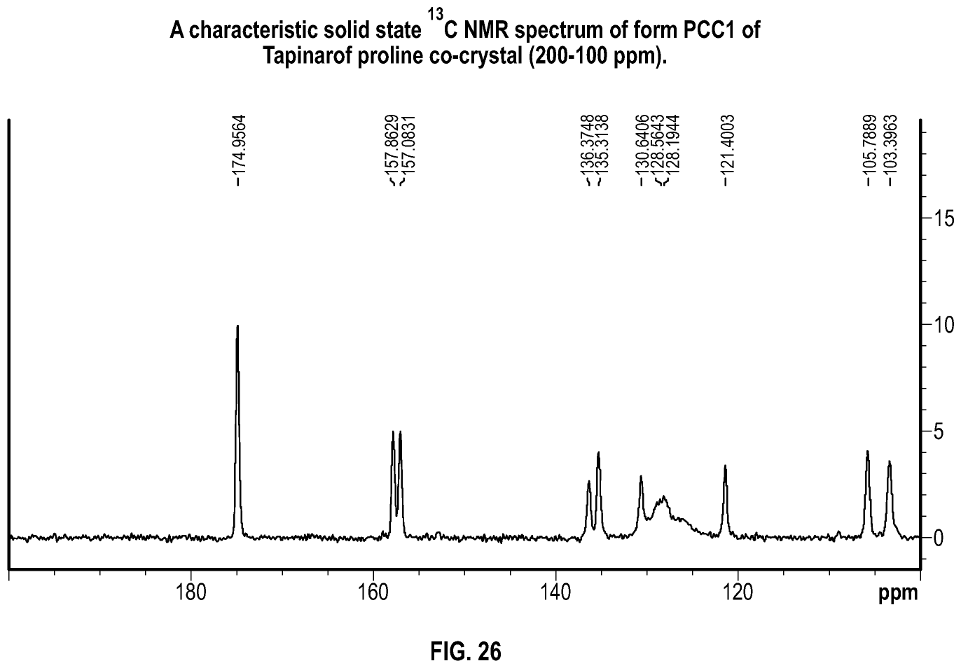

Crystalline form PCC1 of Tapinarof proline may be characterized by data selected from one or more of the following: an X-ray powder diffraction pattern substantially as depicted in FIG. 4; an X-ray powder diffraction pattern having peaks at 6.7, 11.1, 17.8, 19.0 and 23.4 degrees 2-theta±0.2 degrees 2-theta; a solid state ¹³C NMR spectrum with characteristic peaks at 157.9, 157.1, 135.3, 121.4 and 105.8 ppm±2 ppm; a solid state ¹³C NMR spectrum having the following chemical shift absolute differences from reference peak at 175.0 ppm±1 ppm: 17.1, 17.9, 47.7, 53.6, 69.2 ppm±1 ppm; a solid state ¹³C NMR spectrum substantially as depicted in any of FIGS. 24, 25 and 26; and combinations of these data.

Crystalline Form PCC1 of Tapinarof proline may be further characterized by an X-ray powder diffraction pattern having peaks at 6.7, 11.1, 17.8, 19.0 and 23.4 degrees 2-theta±0.2 degrees 2-theta, and also having any one, two, three, four or five additional peaks selected from 13.4, 14.6, 16.0, 19.9 and 22.0 degrees 2-theta±0.2 degrees 2-theta.

Crystalline form PCC1 of Tapinarof proline may be alternatively characterized by an X-ray powder diffraction pattern having peaks at 6.7, 11.1, 13.4, 14.6, 16.0, 17.8, 19.0, 19.9, 22.0 and 23.4 degrees 2-theta±0.2 degrees 2-theta.

Alternatively, or in addition to the above, form PCC1 of Tapinarof proline co-crystal may be characterized by the following unit cell data:
a=13.2669 Å
b=13.2669 Å
c=15.1744 Å
α=90°
β=900
γ=900
cell_volume 1670.9 Å³
symmetry cell setting: tetragonal
Space group P 4₁.

In one embodiment of the present disclosure, crystalline Form PCC1 of Tapinarof proline is isolated.

Crystalline PCC1 of Tapinarof proline co crystal may be characterized by each of the above characteristics alone or by all possible combinations, e.g., an XRPD pattern having peaks at 6.7, 11.1, 17.8, 19.0 and 23.4 degrees 2-theta±0.2 degrees 2-theta; an XRPD pattern as depicted in FIG. 4, and combinations of these data.

The present disclosure further encompasses Tapinarof piperazine co-crystal designated form PCC2. In embodiments the molar ratio of piperazine and Tapinarof in form PCC2 is typically about 1.

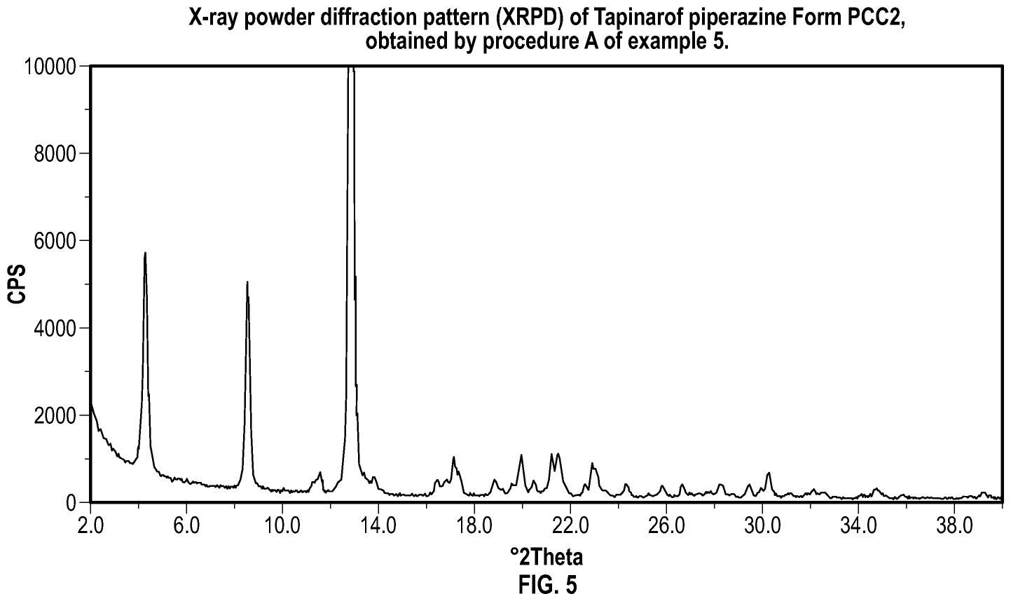

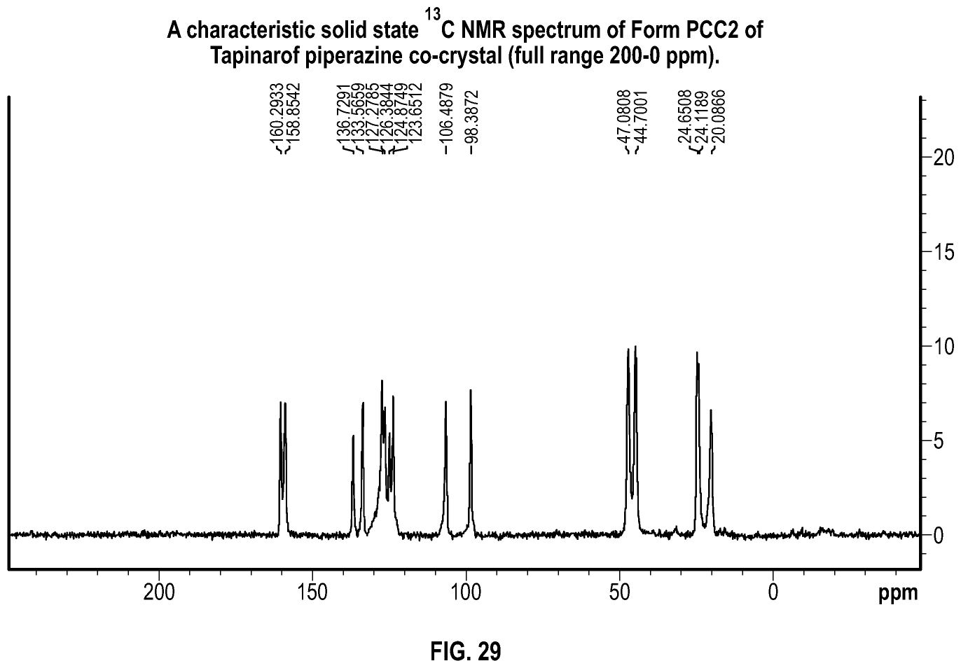

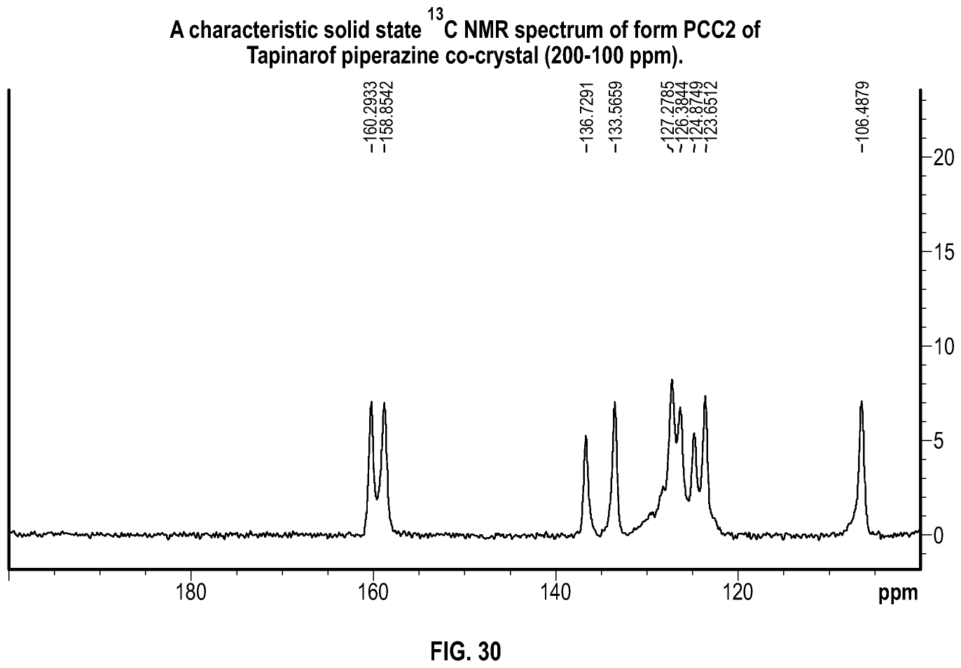

Crystalline form PCC2 of Tapinarof piperazine may be characterized by data selected from one or more of the following: an X-ray powder diffraction pattern substantially as depicted in FIG. 5; an X-ray powder diffraction pattern having peaks at 4.3, 8.5, 12.8, 17.2 and 23.0 degrees 2-theta±0.2 degrees 2-theta; a solid state ¹³C NMR spectrum with characteristic peaks at 160.3, 158.9, 136.7, 133.6 and 123.7 ppm±2 ppm; a solid state ¹³C NMR spectrum having the following chemical shift absolute differences from reference peak at 106.5 ppm±1 ppm: 53.8, 52.4, 30.2, 27.1 and 17.2 ppm±1 ppm; a solid state ¹³C NMR spectrum substantially as depicted in any of FIGS. 29 and 30; and combinations of these data.

Crystalline Form PCC2 of Tapinarof piperazine may be further characterized by an X-ray powder diffraction pattern having peaks at 4.3, 8.5, 12.8, 17.2 and 23.0 degrees 2-theta±0.2 degrees 2-theta, and also having any one, two, three, four or five additional peaks selected from 11.6, 18.9, 20.0, 20.5 and 21.2 degrees 2-theta±0.2 degrees 2-theta.

Crystalline form PCC2 of Tapinarof piperazine may be alternatively characterized by an X-ray powder diffraction pattern having peaks at 4.3, 8.5, 11.6, 12.8, 17.2, 18.9, 20.0, 20.5, 21.2 and 23.0 degrees 2-theta±0.2 degrees 2-theta.

Alternatively, or in addition to the above, form PCC2 of Tapinarof piperazine co-crystal may be characterized by the following unit cell data:
a=26.6603 Å
b=5.5590 Å
c=16.7391 Å
α=90°
β=91.16573°
γ=900
cell_volume 1921.7 Å³
symmetry cell setting: monoclinic
Space group P 2₁/c.

In one embodiment of the present disclosure, crystalline Form PCC2 of Tapinarof piperazine is isolated.

Crystalline PCC2 of Tapinarof piperazine co-crystal may be characterized by each of the above characteristics alone or by all possible combinations, e.g., an XRPD pattern having peaks at 4.3, 8.5, 12.8, 17.2 and 23.0 degrees 2-theta±0.2 degrees 2-theta; an XRPD pattern as depicted in FIG. 5, and combinations of these data.

The above crystalline polymorphs can be used to prepare other crystalline polymorphs of Tapinarof.

The present disclosure encompasses a process for preparing other solid state forms of Tapinarof. The process includes preparing any a solid state form of Tapinarof by the processes of the present disclosure, and converting said form to a different form of Tapinarof.

The present disclosure provides the above described crystalline polymorphs of Tapinarof for use in the preparation of pharmaceutical compositions comprising Tapinarof and/or crystalline polymorphs thereof.

In any aspect or embodiment of the present disclosure, any of the solid state forms described herein, including co-crystal forms and solvate forms, may be polymorphically pure or may be substantially free of any other solid state (or polymorphic) forms. In any aspect or embodiment of the present disclosure, any of the solid state forms described herein including co-crystal forms and solvate forms, may contain: about 20% (w/w) or less, about 10% (w/w) or less, about 5% (w/w) or less, about 2% (w/w) or less, about 1% (w/w) or less, or about 0% of any other forms of the subject compound as measured, preferably by XRPD. Thus, any of crystalline forms of Tapinarof described herein, including Tapinarof co-crystals or Tapinarof solvates, may be substantially free of any other solid state forms, and may contain greater than about 80% (w/w), greater than about 90% (w/w), greater than about 95% (w/w), greater than about 98% (w/w), greater than about 99% (w/w), or about 100% of the subject crystalline form of Tapinarof. In aspect or embodiment of the present disclosure, any of the described crystalline polymorph of Tapinarof may contain: about 0.5% to about 20% (w/w), about 0.5% to about 15% (w/w), about 0.50% to about 10% (w/w), about 0.50% to about 5% (w/w) about 0.5 to about 2% (w/w), about 1% to about 20% (w/w), about 1% to about 10% (w/w), about 1% to about 5%, about 5% to about 20% (w/w), or from about 5% to about 10% (w/w), of one or more other crystalline polymorph of Tapinarof.

In any aspect or embodiment of the present disclosure, the Tapinarof co-crystals may be anhydrous. Particularly, Tapinarof co-crystal as described in any aspect or embodiment of the present disclosure may contain: 1% (w/w) or less, 0.5% (w/w) or less, or 0.2% (w/w) or less, of water or organic solvents, as measured by thermal gravimetric analysis (TGA).

In any aspect or embodiment of the present disclosure, the Tapinarof solvates may be anhydrous. Particularly, Tapinarof solvates as described in any aspect or embodiment of the present disclosure may contain: 1% (w/w) or less, 0.5% (w/w) or less, or 0.2% (w/w) or less, of water or other organic solvents (i.e., organic solvents that are not part of the solvate structure), as measured by thermal gravimetric analysis (TGA).

The present disclosure provides a process for preparation of form T1 of Tapinarof, wherein the process comprises:
a) providing Tapinarof in propionic acid or a mixture of propionic acid and one or more solvents, in embodiments one or more polar solvents such as water;
b) optionally heating the reaction mixture, preferably to a temperature of about 60° C. to about 80° C. to obtain dissolution;
c) optionally cooling to RT;
d) stirring the reaction mixture for about 12-48 hours;
e) optionally separating the precipitate; and
f) optionally washing or drying the precipitate.

In embodiments the starting material provided in step a) is Tapinarof form I or Tapinarof form IV.

In embodiments a slurry is obtained in step (a).

In embodiments the precipitate is separated by centrifuge.

The present disclosure provides a process for preparation of form T1 wherein the process comprises:
a) providing Tapinarof in propionic acid or in a mixture of propionic acid and one or more solvents (optionally wherein the solvent is water);
b) optionally stirring the reaction mixture, for example at room temperature (optionally for about 8 to about 48 hours, about 12 to about 36 hours, or about 12 to about 24 hours);
c) optionally separating the precipitate (optionally by filtration or by centrifuge, optionally by centrifuge); and
d) optionally washing and/or drying the precipitate.

In a particular embodiment, the disclosure provides a process for preparation of form T1 wherein the process comprises:
(a) providing Tapinarof, preferably form I or form IV of Tapinarof in a mixture of propionic acid and water to obtain a slurry;
(b) optionally stirring the reaction mixture (optionally for about 8 to about 48 hours, about 12 to about 36 hours, or about 12 to about 24 hours);
(c) optionally separating the precipitate (optionally by filtration or by centrifuge, optionally by centrifuge);
(d) optionally washing the precipitate; and
(e) optionally drying the precipitate.

Alternatively, the present disclosure provides a process for preparation of form T1 wherein the process comprises:
a) providing Tapinarof in propionic acid or a mixture of propionic acid and one or more solvents (optionally wherein the solvent is water), and heating to obtain dissolution; b) optionally filtering the obtained solution;
c) cooling to a temperature of about 15 to about 30° C.;
d) optionally stirring the reaction mixture (optionally for about 8 to about 48 hours, about 12 to about 36 hours, or about 12 to about 24 hours);
e) optionally separating the precipitate (optionally by filtration or by centrifuge, optionally by filtration); and
f) optionally washing and/or drying the precipitate.

In a particular embodiment, the present disclosure provides a process for preparation of form T1 wherein the process comprises:
(a) providing Tapinarof in propionic acid and heating to a temperature of about 60° C. to about 80° C. to obtain dissolution;
(b) optionally filtering the obtained solution;
(c) cooling to a temperature of about 15 to about 30° C.;
(d) optionally stirring the reaction mixture for about 8 to about 48 hours, about 12 to about 48 hours, about 12 to about 36 hours, or about 12 to about 24 hours);
(e) optionally separating the precipitate (optionally by filtration or by centrifuge, optionally by filtration); and
(f) optionally washing and/or drying the precipitate.

In any of the herein described processes for preparing Form T1 of Tapinarof, the propionic acid may be used in an amount of: about 5 ml to about 30 ml per gram of Tapinarof, about 5 ml to about 20 ml per gram of Tapinarof, about 8 ml to about 18 ml per gram of Tapinarof, or about 10 ml to about 20 ml per gram of Tapinarof.

In any of the herein described processes for preparing Form T1 of Tapinarof, the solvent when present, may be used in 10 ml to about 50 ml per gram of Tapinarof, about 20 ml to about 45 ml per gram of Tapinarof, about 30 ml to about 40 ml per gram of Tapinarof, or about 35 ml per gram of Tapinarof.

In any of the herein described processes for preparing Form T1 of Tapinarof, the mixture in step (a) comprises water and propionic acid in a volume ratio of water:propionic acid of: about 10:1 to about 1:2, about 8:1 to about 1:1, about 5:1 to about 1.5:1, about 3:1 to about 2:1, or about 2.3:1.

In any of the herein described processes for preparing Form T1 of Tapinarof, when the reaction mixture is a slurry, the starting material may be Tapinarof form I or form IV, as described above, and as disclosed in WO 2019/063002.

The present disclosure provides a process for preparation of form T4 of Tapinarof, wherein the processes comprising:
(a) providing Tapinarof in a solution of cyclohexanone or a mixture of cyclohexanone and one or more solvents;
(b) combining the solution with an anti-solvent to obtain a slurry;
(c) optionally stirring the slurry;
(d) optionally separating the precipitate; and
(e) optionally washing or drying the precipitate.

In embodiments, Tapinarof is provided in cyclohexanone as a sole solvent. Optionally the cyclohexanone may be used in an amount of: about 0.5 ml to about 6 ml, about 1 ml to about 5 ml, about −2 ml to about 4 ml, or about 3 ml, per gram of Tapinarof. In other embodiments the reaction mixture in step (a) is heated to a temperature of about 50° C. to about 70° C. to obtain dissolution.

In embodiments, the anti-solvent added in step (b) is a non-polar solvent. preferably a C5-C8 alkane or a cycloalkane, more preferably the anti-solvent is selected from the group consisting of hexane, heptane and cyclohexane to obtain a slurry.

In embodiments the anti-solvent added in step (b) is cooled to a temperature of about 0 to about 10° C. prior to addition to the reaction mixture.

In particular, the present disclosure provides a process for preparation of form T4 wherein the process comprises:
a) providing a mixture of Tapinarof in cyclohexanone and heating the reaction mixture to obtain dissolution, preferably wherein the heating is to a temperature of about 50° C. to about 70° C., or about 60° C.;
b) optionally cooling the solution, preferably to about room temperature;
c) combining the solution with an anti-solvent to obtain a slurry, wherein the antisolvent is preferably selected from the group consisting of: hexane, heptane and cyclohexane, preferably the anti-solvent is heptane; optionally wherein the anti-solvent is cooled to a temperature of about 0 to about 10° C. prior to addition to the reaction mixture;
d) optionally stirring the slurry (optionally for a period of about 30 minutes to about 8 hours, about 45 minutes to about 4 hours, or about 1 hour to about 2 hours);
e) optionally separating the precipitate; and
f) optionally washing and/or drying the precipitate.

The present disclosure provides a process for preparation of form T9 of Tapinarof, wherein the process comprises:
(a) providing Tapinarof in a solution of isoamyl alcohol or a mixture of isoamyl alcohol and one or more solvents;
(b) combining the solution with an anti-solvent to obtain a slurry;
(c) optionally stirring the slurry;
(d) optionally separating the precipitate; and
(e) optionally washing or drying the precipitate.

In embodiments, Tapinarof is provided in isoamyl alcohol as a sole solvent. Optionally, the solution in step (a) may be at room temperature.

In embodiments, the antisolvent added in step (b) is a non-polar solvent. preferably an C5-C8 alkane or a cycloalkane, more preferably the anti-solvent is selected from the group consisting of hexane, heptane and cyclohexane to obtain a slurry.

In embodiments the anti-solvent in step (b) is cooled before addition to the reaction mixture, to a temperature below room temperature (RT), preferably to about 0° C. to about 10° C.

In particular, the present disclosure provides a process for preparation of form T9 of Tapinarof, wherein the process comprises:
a) providing Tapinarof in isoamyl alcohol;
b) stirring to obtain dissolution;
c) combining the solution with an anti-solvent selected from the group consisting of hexane, heptane and cyclohexane, preferably the anti-solvent is heptane, optionally wherein the anti-solvent is precooled to a temp below RT, preferably to about 0° C. to about 10° C. to obtain a slurry;
c) optionally cooling the obtained slurry;
d) optionally stirring the slurry for about 12-48 hours;
e) optionally separating the precipitate; and
f) Optionally washing or drying the precipitate.

In one aspect, the process comprises crystallising Tapinarof nicotinamide co-crystal from a solution comprising Tapinarof and nicotinamide in at least one organic solvent, optionally by cooling and/or addition of an antisolvent. The solvent and anti-solvent may be any of the options described below. Alternatively, the process comprises crystallizing Tapinarof nicotinamide co-crystal from a slurry comprising Tapinarof and nicotinamide, optionally by stirring, typically for about 12 to about 96 hours, to afford the co-crystal.

In one aspect, the present disclosure provides a process for preparation of form NCC1 of Tapinarof nicotinamide co-crystal wherein the process comprises:
(a) providing a solution of Tapinarof and nicotinamide wherein the mole ratio of the ratio of Tapinarof to nicotinamide is about 1:1;
(b) combining the solution with an antisolvent;
(c) optionally stirring;
(d) optionally separating the precipitate; and
(e) optionally washing and/or drying the precipitate.

In embodiments the anti-solvent used in step (b) is pre-cooled to a temperature of about 0 to about 5° C.

In a particular embodiment, the present disclosure provides a process for preparation of form NCC1 of Tapinarof wherein the process comprises:
(a) providing a solution of Tapinarof and nicotinamide in tert-butanol wherein the mole ratio of Tapinarof to nicotinamide is about 1:1;
(b) combining the solution with heptane as anti-solvent, preferably wherein the heptane is—cooled to a temperature of about 0 to about 5° C. prior to addition to the reaction mixture;
(c) optionally stirring;
(d) optionally separating the precipitate; and
(e) optionally washing and/or drying the precipitate.

Alternatively, the present disclosure provides a process for preparation of form NCC1 of Tapinarof nicotinamide co-crystal where the process comprises:
(a) providing a slurry of Tapinarof (optionally form 1 or form IV) and nicotinamide (optionally one molar equivalent) in one or more solvents optionally at a temperature which is greater than room temperature wherein the mole ratio of Tapinarof to nicotinamide is about 1:1;
(b) stirring the reaction mixture for about 12 to about 96 hours optionally at a temperature which is higher than room temperature;
(c) optionally separating the precipitate; and
(d) optionally washing and/or drying the precipitate.

In any embodiment of the process for preparing Tapinarof nicotinamide co-crystal form NCC1, the reaction mixture in step (a) may be a slurry. Optionally, according to any embodiment, the solvent in step (a) is cyclohexane. In embodiments the reaction mixture in steps (a) is heated to a temperature of about 30 to about 50° C. In embodiments the reaction mixture in steps (b) is heated to a temperature of about 30 to about 50° C.

In a particular embodiment the disclosure provides a process for preparation of form NCC1 of Tapinarof nicotinamide co-crystal, wherein the process comprises:
(a) providing Tapinarof and nicotinamide in cyclohexane, wherein the mole ratio of the equivalents of Tapinarof to nicotinamide is about 1:1, optionally at a temperature of about 30 to about 50° C.;
(b) stirring the reaction mixture for about 12 to about 96 hours optionally at a temperature of about 30 to about 50° C.;
(c) optionally separating the precipitate; and
(d) optionally washing and/or drying the precipitate.

The present disclosure provides a process for preparation of form NCC2 of Tapinarof nicotinamide co-crystal where the process comprises:
(a) providing a slurry of Tapinarof (optionally form I or form IV) and nicotinamide in one or more solvents wherein the mole ratio of Tapinarof to nicotinamide is about 1:0.5;
(b) stirring the reaction mixture for about 12 to about 96 hours;
(c) optionally separating the precipitate; and
(d) optionally washing and/or drying the precipitate.

In embodiments the solvent in step a) is toluene. In other embodiments, the solvent is step a) comprises water and an alcohol, preferably the mixture comprises of a water and methanol. In embodiments the ratio of water to methanol is about 10:1 to about 5:1, preferably about 15:2 by volume.

In any embodiment of the processes described herein, the Tapinarof used in step (a) when the mixture is in the form of a slurry, may be form I, IV, T4 or T9.

The present disclosure provides a process for preparation crystalline Tapinarof proline co-crystal, preferably from PCC1, wherein the process comprises:
(a) providing Tapinarof (optionally form I or form IV) and proline in one or more solvents, optionally at a temperature which is higher than room temperature;
(b) stirring the reaction mixture for about 12 to about 96 hours optionally at a temperature which is higher than room temperature;
(c) optionally separating the precipitate; and
(d) optionally washing and/or drying the precipitate.

In embodiments the reaction mixture in step (a) is a slurry. In embodiments the solvent in step (a) is tert-butanol. In embodiments the reaction mixture in steps (a) is heated to a temperature of about 30 to about 50° C. In embodiments the reaction mixture in steps (b) is heated to a temperature of about 30 to about 50° C.

In a particular embodiment the disclosure provides a process for preparation of Tapinarof proline co-crystal, preferably form PCC1, wherein the process comprises:
(a) providing Tapinarof and proline in tert-butanol, optionally at a temperature of about 30 to about 50° C.;
(b) stirring the reaction mixture for about 12 to about 96 hours optionally at a temperature of about 30 to about 50° C.;
(c) optionally separating the precipitate; and
(d) optionally washing and/or drying the precipitate.

In embodiments the Tapinarof used in step a) may be form I, form IV, T4 or T9,

In an alternative embodiment, the present disclosure provides a process for preparation of Tapinarof proline co-crystal, preferably form PCC1, wherein the process comprises:
(a) providing a solution of Tapinarof in one or more organic solvents, optionally at a temperature which is higher than room temperature;
(b) combining the solution with proline optionally wherein the proline is in the form of a suspension;
(c) stirring the reaction mixture for about 12 to about 96 hours optionally at a temperature which is higher than room temperature;
(d) optionally separating the precipitate; and
(e) optionally washing and/or drying the precipitate.

In embodiments step (a) is performed at a temperature of about 30 to about 50° C. In embodiments step (b) is performed at a temperature of about 30 to about 50° C. In embodiments steps (c) is performed at a temperature of about 30 to about 50° C. In particular embodiments, the solvent is tert-butanol.

In particular the present disclosure provides a process for preparation of Tapinarof proline co-crystal, preferably form PCC1, wherein the process comprises:
(a) providing a solution of Tapinarof in tert-butanol, optionally at a temperature of about 30 to about 50° C.;
(b) combining the solution with proline optionally wherein the proline is in the form of a suspension of about 30 to about 50° C.;
(c) stirring the reaction mixture for about 12 to about 96 hours optionally at a temperature of about 30 to about 50° C.;
(d) optionally separating the precipitate; and
(e) optionally washing and/or drying the precipitate.

In another aspect, the process comprises crystallising Tapinarof piperazine co-crystal, preferably form PCC2, from a solution comprising Tapinarof and piperazine in at least one organic solvent, optionally by cooling and/or addition of an antisolvent. The solvent and antisolvent may be any of the options described below. Alternatively, the process comprises crystallizing Tapinarof piperazine co-crystal from a slurry comprising Tapinarof and piperazine, optionally by stirring, typically for about 12 to about 96 hours, to afford the co-crystal.

The present disclosure provides a process for preparation of crystalline Tapinarof piperazine co-crystal, preferably form PCC2, wherein the process comprises:
(a) providing a solution of Tapinarof and piperazine optionally at a temperature higher than room temperature;
(b) optionally filtering the solution;
(c) combining the solution with an anti-solvent, to obtain a slurry;
(d) optionally cooling the obtained slurry;
(e) optionally adding anti-solvent;
(f) optionally stirring the slurry;
(g) optionally separating the precipitate; and
(h) optionally washing and/or drying the precipitate.

In embodiments step (a) is performed at a temperature of about 40 to about −60° C. In embodiments step (b) is performed at a temperature of about 40 to about 60° C. In embodiments steps (c) is performed at a temperature of about 40 to about 60° C. In particular embodiments, the solvent in step (a) is tert-butanol.

In embodiments, the antisolvent added in steps (c) and/or (e) is a non-polar solvent, preferably an C5-C8 alkane or a cycloalkane, more preferably the anti-solvent is selected from the group consisting of hexane, heptane and cyclohexane.

In particular the present disclosure provides a process for preparation of Tapinarof proline co-crystal, preferably form PCC2, wherein the process comprises:
(a) providing a solution of Tapinarof and piperazine in tert-butanol, optionally at a temperature of about 40 to about 60° C.;
(b) combining the solution with heptane optionally at a temperature of about 30 to about 50° C.;
(c) optionally cooling to room temperature;
(d) optionally adding heptane;
(e) optionally separating the precipitate; and
(f) optionally washing and/or drying the precipitate.

Alternatively, the present disclosure provides a process for preparation of form PCC2 of Tapinarof piperazine co-crystal wherein the process comprises:
(a) providing a slurry of Tapinarof and piperazine in one or more solvents optionally at a temperature which is higher than room temperature; (b) optionally cooling the reaction mixture;
(c) optionally stirring the reaction mixture;
(d) optionally separating the precipitate; and
(e) optionally washing and/or drying the precipitate.

In embodiments the reaction mixture in step (a) is a slurry. In embodiments the solvent in step (a) is a mixture of ethyl-acetate and heptane. In a particular embodiment the disclosure provides a process for preparation of Tapinarof piperazine co-crystal, preferably form PCC2, wherein the process comprises:
(a) providing Tapinarof and piperazine in a mixture of ethyl acetate and heptane;
(b) stirring the reaction mixture for about 12 to about 96 hours;
(c) optionally separating the precipitate; and
(d) optionally washing and/or drying the precipitate.

In any embodiment of the processes disclosed herein the drying may be carried out by air (e.g., under filtration), or in a vacuum oven. Optionally drying in a vacuum oven may be carried out at about 20 to about 60° C., or about 30 to about 50° C., or about 30 to about 40° C., or at room temperature.

According to any embodiment of the processes disclosed herein, the process may further comprise combining the resulting solid state form of Tapinarof, with at least one pharmaceutically acceptable excipient, to form a pharmaceutical composition, preferably for topical administration.

Any one of the above described solid state forms of Tapinarof or co-crystals of Tapinarof may be used for preparation of other solid state forms.

The present disclosure also encompasses the use of crystalline polymorphs of Tapinarof or co-crystals of Tapinarof of the present disclosure for the preparation of pharmaceutical compositions of any one or a combination of the crystalline polymorphs Tapinarof and/or crystalline polymorphs thereof.

The present disclosure includes processes for preparing the above mentioned pharmaceutical compositions. The processes include combining any one or a combination of the crystalline polymorphs of Tapinarof or the co-crystals of Tapinarof of the present disclosure with at least one pharmaceutically acceptable excipient.

Pharmaceutical combinations or formulations of the present disclosure contain any one or a combination of the solid state forms of Tapinarof or co-crystals of Tapinarof of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Tapinarof and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Tapinarof can be administered. Tapinarof may be formulated for administration to a mammal, in embodiments to a human, by injection. Tapinarof can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

Tapinarof can be administered by a topical formulation. Thus, pharmaceutical compositions of the present invention may particularly be in a form that is suitable for topical application, for example, a cream, an ointment, a solution, or a lotion. The topical formulation may comprise any one or more of an oil phase, a water phase, surfactants, antioxidants, pH adjusting agents, gelling agents, preservatives and/or other dermatologically acceptable excipients.

The crystalline polymorphs of Tapinarof including the co-crystals of Tapinarof and the pharmaceutical compositions and/or formulations of Tapinarof of the present disclosure can be used as medicaments, in embodiments in the treatment of psoriasis and atopic dermatitis.

The present disclosure also provides methods of treating psoriasis and atopic dermatitis by administering a therapeutically effective amount of any one or a combination of the crystalline polymorphs of Tapinarof and/or the co-crystals of Tapinarof of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

XRPD analysis was performed on ARL (SCINTAG) powder X-Ray diffractometer model X'TRA equipped with a solid state detector. Copper radiation of 1.5418 Å was used. Scanning parameters: range: 2-40 degrees two-theta; scan mode: continuous scan; step size: 0.05°, and a rate of 3 deg/minute.

The analysis was done using silicon powder as an internal standard; the position of the silicon (Si) peak was corrected in accordance with silicone theoretical peak: 28.45 degrees two theta.

### Solid-State NMR

Solid-state NMR spectra were measured at 11.7 T using a Bruker Avance III HD 500 US/WB NMR spectrometer (Karlsruhe, Germany, 2013). The 13C CP/MAS NMR spectra employing cross-polarization were acquired using the standard pulse scheme at spinning frequency of 18 kHz. The recycle delay was 8 s and the cross-polarization contact time was 2 ms. The strength of spin-locking fields B1(13C) expressed in frequency units ω½ π=γ B1 was 64 kHz.

### SEM Method

SEM micrographs were taken on Phenom Pro, scanning microscope at 10 kV, low current. Samples were sputtered with gold by Denton Desk V sputter coater.

### TGA Method

Thermogravimetric analysis was carried out Mettler Toledo TGA/DSC with the following scanning parameters:
Heating between 25-250° C.
Heating rate: 10° C./minute.
Purging with 40 ml/min N₂ flow.
Sample weight: 7-15 mg.
Crucible: 150 μL alumina Crucible with standard aluminum lid.

### Unit Cell Dimensions Method

The powder data for structure determination were collected at room temperature on Rigaku powder diffractometer with Cu Kα1 radiation and primary monochromitor. Sample was placed in a rotation capillary. The structure solution was done in DASH software.

### Molecular Model

The molecular model of Tapinarof was created by molecular modelling, and energy minimized by PM3 semi-empirical QM method. Final data were exported to the cif format.

### EXAMPLES

### Preparation of Starting Materials

Tapinarof Form IV was prepared according to the procedure described in International Publication No. WO 2019/063002 or according to reference example 1.

### Reference Example 1—Preparation of Form IV Tapinarof

Toluene (3 ml, 6V) was added to Tapinarof (500 mg, 2 mmol) to obtain slurry. The slurry was magnetically stirred and heated to 50° C. over a period of 15 minutes to obtain complete dissolution followed by filtration using filter disk. The solution was cooled to room temperature and the precipitation occurred. The obtained slurry was stirred at room temperature during 18 hours. The precipitated solid was then separated by Buchner to afford white wet solid and dried in a vacuum oven at 45° C. over a period of 20 hours to afford white solid. The obtained solid was analyzed by X-ray powder diffraction and identified as Tapinarof form IV and the XRPD pattern obtained is presented in FIG. 6.

### Reference Example 2—Preparation of Form IV Tapinarof

Methanol (0.5 ml, 10V) was added to Tapinarof (50 mg, 0.2 mmol) to obtain a slurry. The slurry was magnetically stirred at room temperature over a period of 10 minutes to obtain complete dissolution. Next, cold water (4° C.) as anti-solvent (0.5 ml) was added drop-wise to the stirred clear solution at room temperature to obtain solid precipitation. Then, the obtained precipitate was stirred at room temperature during 16 hours. The obtained solid was then isolated by Buchner to afford a white wet solid that was dried in a vacuum oven at 45° C. over a period of 16 hours. The solid was characterized by X-ray powder diffraction as Tapinarof crystal form I and the XRPD pattern obtained is presented in FIG. 13.

### Example 1: Preparation of Tapinarof Crystalline Form T1

### Procedure A

Mixture of water (7 ml, 35V) and propionic acid (3 ml, 15V) was added to Tapinarof Form IV (that was prepared according to the procedure described in WO 2019/063002) (200 mg, 0.8 mmol) to obtain a slurry. The slurry was magnetically stirred at room temperature over a period of 20 hours. The solid was then separated by centrifuge to afford white wet solid and dried in a vacuum oven at RT ° C. over a period of 2.5 hours to afford white solid. The obtained solid was characterized by X-ray powder diffraction and the XRPD pattern is presented in FIG. 1.

### Procedure B

Propionic acid (0.5 ml, 10V) was added to Tapinarof (50 mg, 0.2 mmol) to obtain a slurry. The slurry was magnetically stirred and heated to 70° C. over a period of 15 minutes to obtain complete dissolution followed by filtration using filter disk. Then, the solution was cooled to room temperature and then stirred at room temperature during 16 hours. The obtained precipitate was then separated by centrifuge to afford a white wet solid. The obtained solid analyzed by XRPD and identified as crystal form T1.

### Procedure C

Mixture of water (3.5 ml, 35V) and propionic acid (1.5 ml, 15V) was added to Tapinarof Form I (that may be obtained according to reference example 2, 100 mg, 0.4 mmol) to obtain slurry. The slurry was magnetically stirred at room temperature over a period of 20 hours. The solid was then separated by centrifuge to afford white wet solid that was dried in a vacuum oven at room temperature over a period of 2.5 hours to afford a white solid. The obtained solid was analyzed by X-ray powder diffraction and identified as Tapinarof crystal form T1.

### Example 2: Preparation of Tapinarof Nicotinamide Co-Crystal Form NCC1

### Procedure A

T-butanol (7 ml, 10V) was added to Tapinarof (100 mg, 0.4 mmol) and nicotinamide (48 mg, 0.4 mmol) to obtain slurry. The slurry was magnetically stirred at RT over a period of 15 minutes to obtain complete dissolution. Next, cold heptane as anti-solvent (3 ml) was added drop-wise to the stirred clear solution at RT and then the precipitation occurred. The obtained slurry was stirred at room temperature during 18 hours. The precipitated solid was then separated by Buchner to afford white wet solid and dried in a vacuum oven at 45° C. over a period of 3.5 hours to afford white solid. The obtained solid was analyzed by X-ray powder diffraction and the XRPD pattern is presented in FIG. 2.

### Procedure B

Tapinarof Form IV (50 mg, 0.2 mmol) and nicotinamide (48 mg, 0.39 mmol) was grinded with 2 drops of water during 1 minute at room temperature. The obtained solid was analyzed by X-ray powder diffraction and identified as Tapinarof nicotinamide crystal form NCC1.

### Procedure C

T-butanol (7 ml, 10V) was added to Tapinarof form I (that may be obtained according to reference example 2, 100 mg, 0.4 mmol) and nicotinamide (48 mg, 0.4 mmol) to obtain a slurry. The slurry was magnetically stirred at room temperature over a period of 15 minutes to obtain complete dissolution. Next, cold heptane as anti-solvent (3 ml) was added drop-wise to the stirred clear solution at room temperature and then the precipitation occurred. The obtained slurry was stirred at room temperature during 18 hours. The precipitated solid was then separated by Buchner to afford a white wet solid that was dried in a vacuum oven at 45° C. over a period of 16 hours to afford a white solid. The obtained solid was analyzed by X-ray powder diffraction and identified as Tapinarof nicotinamide crystal form NCC1.

### Procedure D

Cyclohexane (1 ml, 20V) was added to Tapinarof Form IV (that may be obtained according to reference example 1, 50 mg, 0.2 mmol) and nicotinamide (24 mg, 0.2 mmol) to obtain slurry. The slurry was magnetically stirred at 40° C. over a period of 18 hours. The obtained solid was then separated by centrifuge to afford white wet solid and dried in a vacuum oven at 45° C. over a period of 72 hours to afford white solid. The obtained solid was analyzed by X-ray powder diffraction and identified as Tapinarof nicotinamide crystal form NCC1.

### Example 3: Preparation of Tapinarof Nicotinamide Co-Crystal Form NCC2

### Procedure A

Toluene (2 ml, 20V) was added to Tapinarof Form IV (100 mg, 0.4 mmol) and nicotinamide (48 mg, 0.4 mmol) to obtain slurry. The slurry was magnetically stirred at RT over a period of 72 hours. The obtained solid was dried in a vacuum oven at 45° C. over a period of 16 hours to afford white solid. The obtained solid was analyzed by X-ray powder diffraction and the XRPD pattern is presented in FIG. 3.

### Procedure B

Mixture of water (0.75 ml, 15V) and MeOH (0.1 ml, 2V) was added to Tapinarof Form IV (50 mg, 0.2 mmol) and nicotinamide (48 mg, 0.4 mmol) to obtain slurry. The slurry was magnetically stirred at RT over a period of 48 hours. The obtained solid was then separated by centrifuge to afford white wet solid and dried in a vacuum oven at 45° C. over a period of 16 hours to afford white solid. The obtained solid was analyzed by X-ray powder diffraction and identified as Tapinarof nicotinamide crystal form NCC2.

### Procedure C

Toluene (2 ml, 20V) was added to Tapinarof form I (that can be prepared according to reference example 2) (100 mg, 0.4 mmol) and nicotinamide (24 mg, 0.2 mmol) to obtain a slurry. The slurry was magnetically stirred at room temperature over a period of 18 hours. The obtained solid was then separated by centrifuge to afford white wet solid that was dried in a vacuum oven at 45° C. over a period of 16 hours to afford white solid. The obtained solid was analyzed by X-ray powder diffraction and identified as Tapinarof nicotinamide crystal form NCC2.

### Procedure D

Toluene (0.9 ml, 17.5V) was added to Tapinarof form T9 (that may be obtained according to example 11, 50 mg, 0.2 mmol) and nicotinamide (12 mg, 0.2 mmol) to obtain a slurry. The slurry was magnetically stirred at room temperature over a period of 72 hours. The obtained solid was then separated by centrifuge to afford white wet solid and was analyzed by X-ray powder diffraction and identified as Tapinarof nicotinamide crystal form NCC2.

### Procedure E

Toluene (0.5 ml, 10V) was added to Tapinarof form T4 (that may be obtained according to example 8, 50 mg, 0.2 mmol) and nicotinamide (12 mg, 0.2 mmol) to obtain a slurry. The slurry was magnetically stirred at room temperature over a period of 18 hours. The obtained solid was then separated by centrifuge to afford white wet solid and was analyzed by X-ray powder diffraction and identified as Tapinarof nicotinamide crystal form NCC2.

### Example 4: Preparation of Tapinarof Proline Co-Crystal Form PCC1

### Procedure A

T-butanol (1.5 ml, 15V) was added to Tapinarof Form IV (100 mg, 0.4 mmol) and proline (91 mg, 0.8 mmol) to obtain slurry. The slurry was magnetically stirred at 40° C. over a period of 18 hours. The obtained solid was then separated by Buchner to afford white wet solid and dried in a vacuum oven at 45° C. over a period of 20 hours to afford white solid. The obtained solid was analyzed by X-ray powder diffraction and the XRPD pattern is presented in FIG. 4.

### Procedure B

Tapinarof Form IV (50 mg, 0.2 mmol) and proline (48 mg, 0.42 mmol) was grinded with 2 drops of Isopropyl alcohol during 1 minute at room temperature. The obtained solid was analyzed by X-ray powder diffraction and identified as Tapinarof proline crystal form PCC1. Procedure C

T-butanol (1.5 ml, 15V) was added to Tapinarof Form I (that can be prepared according to reference example 2) (100 mg, 0.4 mmol) and proline (22.5 mg, 0.2 mmol) to obtain slurry. The slurry was magnetically stirred at 40° C. over a period of 18 hours. The obtained solid was then separated by Buchner to afford white wet solid that was dried in a vacuum oven at 45° C. over a period of 16 hours to afford a white solid. The obtained solid was analyzed by X-ray powder diffraction and identified as Tapinarof proline crystal form PCC1.

### Procedure D

T-butanol (0.4 ml, 7.5V) was added to Tapinarof Form T9 (that can be prepared according to example 11) (50 mg, 0.2 mmol) and proline (11.5 mg, 0.1 mmol) to obtain a slurry. The slurry was magnetically stirred at 40° C. over a period of 72 hours. The obtained solid was then separated by Buchner to afford white wet solid. The obtained solid was analyzed by X-ray powder diffraction and identified as Tapinarof proline crystal form PCC1.

### Procedure E

T-butanol (0.4 ml, 7.5V) was added to Tapinarof Form T4 (that can be prepared according to example 8) (50 mg, 0.2 mmol) and proline (11.5 mg, 0.1 mmol) to obtain a slurry. The slurry was magnetically stirred at 40° C. over a period of 18 hours. The obtained solid was then separated by Buchner to afford a white wet solid. The obtained solid was analyzed by X-ray powder diffraction and identified as Tapinarof proline crystal form PCC1.

### Example 5: Preparation of Tapinarof Piperazine Co-Crystal Form PCC2

### Procedure A

T-butanol (10 ml, 20V) was added to Tapinarof (500 mg, 2 mmol) and piperazine (170 mg, 2 mmol) to obtain slurry. The slurry was magnetically stirred at 40° C. over a period of 15 minutes. The obtained light slurry was heated to 50° C. to obtain complete dissolution and after dissolution mechanically filtered using a filter disk. Next, cold heptane as anti-solvent (10 ml) was added drop-wise to the stirred clear solution at 50° C. Then the solution was cooled to room temperature and additional heptane as anti-solvent (5 ml) was added drop-wise to the stirred clear solution at RT and then the precipitation occurred. The obtained slurry was stirred at room temperature during 18 hours. The precipitated solid was then separated by Buchner to afford white wet solid and dried in a vacuum oven at 45° C. over a period of 72 hours to afford white solid. The obtained solid was analyzed by X-ray powder diffraction and the XRPD pattern is presented in FIG. 5.

### Procedure B

T-butanol (10 ml, 20V) was added to Tapinarof form I (that may be obtained according to reference example 2, 500 mg, 2 mmol) and piperazine (170 mg, 2 mmol) to obtain a slurry. The slurry was magnetically stirred at 50° C. over a period of 15 minutes. The obtained light slurry was heated to 50° C. to obtain complete dissolution followed by a hot mechanical filtration using filter disk. Next, cold heptane as anti-solvent (3 ml) was added drop-wise to the stirred clear solution at 50° C. Then the solution was cooled to room temperature and heptane as anti-solvent (5 ml) was added drop-wise to the stirred clear solution at RT and then the precipitation occurred. The obtained slurry was stirred at room temperature during 18 hours. The precipitated solid was then separated by Buchner to afford a white wet solid that was dried in a vacuum oven at 45° C. over a period of 16 hours to afford a white solid. The obtained solid was analyzed by X-ray powder diffraction and identified as Tapinarof piperazine crystal form PCC2.

### Procedure C

Mixture of Ethyl acetate:heptane (1:3) (1 ml, 20V) was added to Tapinarof Form IV (50 mg, 0.2 mmol) and piperazine (17 mg, 0.2 mmol) to obtain slurry. The slurry was magnetically stirred at room temperature over a period of 18 hours. The obtained solid was then separated by centrifuge to afford white wet solid. The obtained solid was analyzed by X-ray powder diffraction and identified as Tapinarof piperazine crystal form PCC2.

### Example 6: Preparation of Tapinarof Form T2

### Procedure A

Dimethyl sulfoxide (1 ml, 2V) was added to Tapinarof (500 mg, 2 mmol) to obtain a slurry. The slurry was magnetically stirred at room temperature over a period of 10 minutes to obtain complete dissolution. Next, cold water (4° C.) as anti-solvent (3 ml) was added drop-wise to the stirred clear solution at room temperature to obtain solid precipitation. Then, the obtained slurry was stirred at room temperature during 24 hours. The obtained solid was then separated by centrifuge to afford a white wet solid and was dried in vacuum oven at 25° C. during 16 hours. The obtained solid was analyzed by X-ray powder diffraction and the XRPD pattern is presented in FIG. 7.

### Example 7: Preparation of Tapinarof Form T3

### Procedure A

N-Methyl-2-pyrrolidone (3 ml, 3V) was added to Tapinarof (1000 mg, 4 mmol) to obtain a slurry. The slurry was magnetically stirred at room temperature over a period of 10 minutes to obtain complete dissolution. Next, cold water (4° C.) as anti-solvent (12 ml) was added drop-wise to the stirred clear solution at room temperature to obtain solid precipitation. Then, the obtained slurry was stirred at room temperature during 16 hours. The obtained solid was then isolated by centrifuge to afford a white wet solid and was dried in vacuum oven at 45° C. during 18 hours. The obtained solid was analyzed by X-ray powder diffraction and the XRPD pattern is presented in FIG. 8.

### Example 8: Preparation of Tapinarof Form T4

### Procedure A

Cyclohexanone (1.8 ml, 6V) was added to Tapinarof (300 mg, 1.2 mmol) to obtain a slurry. The slurry was magnetically stirred at 60° C. over a period of 10 minutes to obtain complete dissolution. Next, the solution was cooled to room temperature and cold heptane (4° C.) as anti-solvent (12 ml) was added drop-wise to the stirred clear solution at room temperature to obtain solid precipitation. Then, the obtained slurry was stirred at room temperature during 1.5 hours. The obtained solid was then isolated by Buchner to afford a white wet solid. The solid was analyzed by X-ray powder diffraction and confirmed to be Form T4 (as per FIG. 9).

### Procedure B

Cyclohexanone (0.9 ml, 3V) was added to Tapinarof (300 mg, 1.2 mmol) to obtain a slurry. The slurry was magnetically stirred at 60° C. over a period of 10 minutes to obtain complete dissolution. Next, the solution was cooled to room temperature and cold heptane (4° C.) as anti-solvent (12 ml) was added drop-wise to the stirred clear solution at room temperature to obtain solid precipitation. Then, the obtained slurry was stirred at room temperature during 1.5 hours. The obtained solid was then isolated by Buchner to afford a white wet solid. The solid was analyzed by X-ray powder diffraction and confirmed to be Form T4 (as per FIG. 9).

### Example 9: Preparation of Tapinarof Form T5

### Procedure A

Acetyl acetone (0.3 ml, 3V) was added to Tapinarof (100 mg, 0.4 mmol) to obtain a slurry. The slurry was magnetically stirred at 60° C. over a period of 10 minutes to obtain complete dissolution. Next, the solution was cooled to room temperature and cold cyclohexane (4° C.) as anti-solvent (1.2 ml) was added drop-wise to the stirred clear solution at room temperature to obtain solid precipitation. Then, the obtained slurry was stirred at room temperature during 16 hours. The obtained solid was then isolated by Buchner to afford a white wet solid. The solid was analyzed by X-ray powder diffraction and the XRPD pattern is presented in FIG. 10.

### Example 10: Preparation of Tapinarof Form T7

### Procedure A

Dimethyl sulfoxide (0.2 ml, 2V) was added to Tapinarof (100 mg, 0.4 mmol) to obtain a slurry. The slurry was magnetically stirred at room temperature over a period of 10 minutes to obtain complete dissolution. Next, cold heptane (4° C.) as anti-solvent (1.2 ml) was added drop-wise to the stirred clear solution at room temperature and then the solution was cooled to 5° C. to obtain solid precipitation. Then, the obtained slurry was stirred at 5° C. during 16 hours. The obtained solid was then isolated by Buchner to afford a white wet solid. The solid was analyzed by X-ray powder diffraction and the XRPD pattern is presented in FIG. 11.

### Example 11: Preparation of Tapinarof Form T9

### Procedure A

Isoamyl alcohol (1 ml, 2V) was added to Tapinarof (500 mg, 2 mmol) to obtain a slurry. The slurry was magnetically stirred at room temperature over a period of 10 minutes to obtain complete dissolution. Next, cold heptane (4° C.) as anti-solvent (5 ml) was added drop-wise to the stirred clear solution at room temperature and then the solution was cooled to 5° C. to obtain solid precipitation. Then, the obtained slurry was stirred at 5° C. during 16 hours. The obtained solid was then isolated by Buchner to afford a white wet solid. The solid was analyzed by X-ray powder diffraction and the XRPD pattern is presented in FIG. 12.

### Example 12: Unit Cell Dimensions of Form NCC1

| Unit cell parameters | Rietveld refinement | |
|---|---|---|
| cell_length_a | 5.1850 | \acute{Å} |
| cell_length_b | 30.6936 | \acute{Å} |
| cell_length_c | 16.5912 | \acute{Å} |
| cell_angle_alpha | 90° | |
| cell_angle_beta | 92.2897 | |
| cell_angle_gamma | 90° | |
| cell_volume | 2638.3 | \acute{Å}³ |

| | |
|---|---|
| symmetry_cell_setting | monoclinic |
| symmetry_space_group_name | Cc |
| cell_measurement_temperature | RT |

### Example 13: Unit Cell Dimensions of Form NCC2

| Unit cell parameters | Rietveld refinement | |
|---|---|---|
| cell_length_a | 15.4014 | \acute{Å} |
| cell_length_b | 5.2492 | \acute{Å} |
| cell_length_c | 28.5194 | \acute{Å} |
| cell_angle_alpha | 90° | |
| cell_angle_beta | 120.1912° | |
| cell_angle_gamma | 90° | |
| cell_volume | 1992.9 | \acute{Å}³ |

| | |
|---|---|
| symmetry_cell_setting | monoclinic |
| symmetry_space_group_name | P 2₁/c |
| cell_measurement_temperature | RT |

### Example 14: Unit Cell Dimensions of Form PCC1

| Unit cell parameters | Rietveld refinement | |
|---|---|---|
| cell_length_a | 13.2669 | \acute{Å} |
| cell_length_b | 13.2669 | \acute{Å} |
| cell_length_c | 15.1744 | \acute{Å} |
| cell_angle_alpha | 90° | |
| cell_angle_beta | 90° | |
| cell_angle_gamma | 90° | |
| cell_volume | 2670.9 | \acute{Å}³ |

| | |
|---|---|
| symmetry_cell_setting | tetragonal |
| symmetry_space_group_name | P 4₁ |
| cell_measurement_temperature | RT |

### Example 15: Unit Cell Dimensions of Form PCC2

| Unit cell parameters | Rietveld refinement | |
|---|---|---|
| cell_length_a | 20.6603 | \acute{Å} |
| cell_length_b | 5.5590 | \acute{Å} |
| cell_length_b | 5.5590 | \acute{Å} |
| cell_length_c | 16.7391 | \acute{Å} |
| cell_angle_alpha | 90° | |
| cell_angle_beta | 91.6573° | |
| cell_angle_gamma | 90° | |
| cell_volume | 1921.7 | \acute{Å}³ |

| | |
|---|---|
| symmetry_cell_setting | monoclinic |
| symmetry_space_group_name | P 21/c |
| cell_measurement_temperature | RT |

1-12. (canceled)

13. Tapinarof nicotinamide co-crystal.

14. Tapinarof nicotinamide co-crystal according to claim 13, designated NCC1, which is characterized by data selected from one or more of the following:
(i) an X-ray powder diffraction pattern substantially as depicted in FIG. 2;
(ii) an X-ray powder diffraction pattern having peaks at 5.8, 7.9, 12.7, 15.8 and 21.4 degrees 2-theta±0.2 degrees 2-theta;
(iii) a solid state ¹³C NMR spectrum with characteristic peaks at 152.1, 144.5, 136.2, 118.0 and 105.1 ppm 2 ppm;
(iv) a solid state ¹³C NMR spectrum having the following chemical shift absolute differences from reference peak at 170.4 ppm±1 ppm: 18.3, 25.9, 34.2, 52.4 and 65.3 ppm±1 ppm;
(v) a solid state ¹³C NMR spectrum substantially as depicted in any of FIGS. 14, 15 and 16; and/or
(vi) a unit cell having the following parameters:
a=5.1850 Å, b=30.6936 Å, c=16.5912 Å;
β=92.2897°;
a monoclinic symmetry cell setting; and space group Cc.

15. Tapinarof nicotinamide co-crystal according to claim 13, which is characterized by an X-ray powder diffraction pattern having peaks at 5.8, 7.9, 12.7, 15.8 and 21.4 degrees 2-theta±0.2 degrees 2-theta, and also having any one, two, three, four or five additional peaks selected from 11.5, 12.1, 17.4, 23.3 and 27.2 degrees 2-theta±0.2 degrees 2-theta.

16. Tapinarof nicotinamide co-crystal according to claim 13, which is characterized by an X-ray powder diffraction pattern having peaks at 5.8, 7.9, 11.5, 12.1, 12.7, 15.8, 17.4, 21.4, 23.3 and 27.2 degrees 2-theta±0.2 degrees 2-theta.

17. Tapinarof nicotinamide co-crystal according to claim 13, wherein the molar ratio of nicotinamide and Tapinarof is about 2:1.

18. Tapinarof nicotinamide co-crystal according to claim 13, designated NCC2, which is characterized by data selected from one or more of the following:
(i) an X-ray powder diffraction pattern having peaks at 6.9, 13.3, 13.8, 21.6 and 22.2 degrees 2-theta±0.2 degrees 2-theta;
(ii) an X-ray powder diffraction pattern substantially as depicted in FIG. 3;
(iii) a solid state ¹³C NMR spectrum with characteristic peaks at 151.5, 147.4, 119.8, 109.7 and 101.5 ppm 2 ppm;
(iv) a solid state ¹³C NMR spectrum having the following chemical shift absolute differences from reference peak at 170.0 ppm±1 ppm: 18.5, 22.6, 50.2, 60.3 and 68.5 ppm±1 ppm;
(v) a solid state ¹³C NMR spectrum substantially as depicted in any of FIGS. 19, 20 and 21; and/or
(vi) a unit cell having the following parameters:
a=15.4014 Å, b=5.2492 Å, c=28.5194 Å;
β=120.1912°;
a monoclinic symmetry cell setting; and space group P 2₁/c.

19. Tapinarof nicotinamide co-crystal according to claim 13, which is characterized by an X-ray powder diffraction pattern having peaks at 6.9, 13.3, 13.8, 21.6 and 22.2 degrees 2-theta±0.2 degrees 2-theta, and also having any one, two, three, four or five additional peaks selected from 12.0, 18.0, 18.3, 24.8 and 25.7 degrees 2-theta±0.2 degrees 2-theta.

20. Tapinarof nicotinamide co-crystal according to claim 13, which is characterized by an X-ray powder diffraction pattern having peaks at 6.9, 12.0, 13.3, 13.8, 18.0, 18.3, 21.6, 22.2, 24.8 and 25.7 degrees 2-theta±0.2 degrees 2-theta.

21. Tapinarof nicotinamide co-crystal according to claim 13, wherein the molar ratio of nicotinamide and Tapinarof is about 1:1.

22. Tapinarof nicotinamide co-crystal according to claim 13, which is isolated, or which is substantially free of any other solid state forms.

23. A Tapinarof co-crystal according to claim 13, which is anhydrous.

24-47. (canceled)

48. A pharmaceutical composition comprising a product according to claim 13 and at least one pharmaceutically acceptable excipient.

49. (canceled)

50. A process for preparing a pharmaceutical composition, comprising combining a product according to claim 13 with at least one pharmaceutically acceptable excipient.

51. A medicament comprising the product according to claim 13.

52. (canceled)

53. A method of treating psoriasis or atopic dermatitis, comprising administering a therapeutically effective amount of a crystalline product according to claim 13, to a subject in need of the treatment.
